(19) United States
(12) Patent Application Publication
(10) Pub. No.: US 2012/0077989 A1
(43) Pub. Date: Mar. 29, 2012

(54) CYCLIC IMIDATE LIGANDS

(76) Inventors: Timothy Noël, De Pinte (BE); Koen Vandyck, Paal-Beringen (BE); Johan Van Der Eycken, Ninove (BE)

(21) Appl. No.: 13/138,842

(22) PCT Filed: Apr. 6, 2010
(86) PCT No.: PCT/EP2010/054549

§ 371 Date: Dec. 7, 2011

(30) Foreign Application Priority Data
Apr. 6, 2009 (GB) 0905995.7

(51) Int. Cl. C07F 15/00; C07D 407/14; C07F 1/08; C07F 9/6568; C07D 405/12; C07D 407/12; C07F 17/02

(52) U.S. Cl. 548/525; 549/467; 549/448; 549/209; 549/220; 549/212

(57) ABSTRACT

The present invention relates to a use of a cyclic imidate as a ligand for catalysis in which the ligand contains sub-structure (Y) as a minimal structural motive, wherein the carbon atoms and the nitrogen atom can be optionally substituted by a chemical substituent.

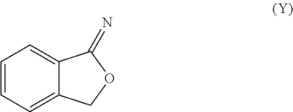

### TECHNICAL FIELD

The present invention relates to the development of novel cyclic imidate ligands and the development of metal complexes thereof, as well as to their synthesis and use in asymmetric catalysis.

### BACKGROUND

Molecular chirality plays an important role in science and technology. The biological activities of many pharmaceuticals, fragrances, food additives and agrochemicals are often associated with their absolute molecular configuration. While one enantiomer displays a desired biological activity through interactions with natural binding sites, the other enantiomer usually does not have the same function and sometimes has deleterious side effects.

A growing demand in industry is to make chiral compounds in enantiomerically pure form. To meet this fascinating challenge, chemists have explored many approaches for acquiring enantiomerically pure compounds ranging from optical resolution and structural modification of naturally occurring chiral substances to asymmetric catalysis using synthetic chiral catalysts and enzymes. Among these methods, asymmetric catalysis is perhaps the most efficient because a small amount of a chiral catalyst can be used to produce a large quantity of a chiral target molecule.

During the last decades, much attention has been devoted to discovering new asymmetric catalysts, and more than half a dozen commercial industrial processes have used asymmetric catalysis as the key step in the production of enantiomerically pure compounds.

Many chiral phosphines have been made to facilitate asymmetric reactions. Among these ligands, 2,2′-bis(diphenylphosphino)-1,1′-binaphthyl “BINAP” is one of the most frequently used bidentate chiral phosphines. The axially dyssymmetric, fully aromatic BINAP ligand has been demonstrated to be highly effective for many asymmetric reactions. DuPHOS and related ligands have also shown impressive enantioselectivities in numerous reactions. However, these phosphines are difficult to make and some of them are air sensitive.

The dramatic growth of enantioselective catalysis results in a permanent search for new chiral ligands. Nitrogen-containing ligands are known as cheap, easily accessible and stable alternatives for phosphines. As a result, a lot of attention has been devoted to the design, synthesis and application of a wide variety of nitrogen ligands such as oxazolines, diimines, semicorrins, 2,2′-bipyridines, pyrrolyl-, pyrrolidinyl-, and pyridyloxazolines, benzoxazines, amidines and sulfoximines. Imidates have, to the best of our knowledge, never been used as ligands in asymmetric catalysis. This is most probably due to their general assumed instability (Ref. 1).

There remains a need in the art for improved ligands, which overcome at least some of the above-mentioned problems.

### SUMMARY

In accordance with the current invention, it was found that imidate compounds solve at least some of these problems.

The present invention provides a use of a cyclic imidate as a ligand for catalysis in which the ligand contains substructure (Y) as a minimal structural motive, wherein the carbon atoms and the nitrogen atom can be optionally substituted by a chemical substituent.

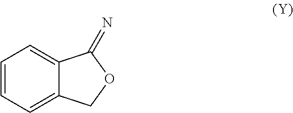

In an embodiment of the invention, the ligand is used in the synthesis of chiral non-racemic building blocks for pharmaceuticals, agrochemicals, flavors and/or fragrances.

In an embodiment of the invention, the ligand is used in the synthesis of achiral or racemic building blocks for organic syntheses.

In an embodiment of the use according to the invention, the cyclic imidate is a cyclic imidate of formula (I), or a stereoisomeric form thereof or a salt thereof,

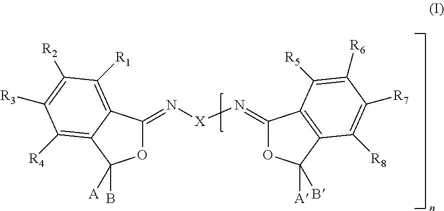

wherein
R₁, R₂, R₃, R₄, R₅, R₆, R₇, and R₈ are each independently selected from the group comprising hydrogen, halogen, alkyl, heteroalkyl, aryl, heteroaryl, hydroxyl, optionally substituted amino, diarylphosphanyl, diheteroarylphosphanyl, arylalkylphosphanyl, heteroarylalkylphosphanyl and dialkylphosphanyl
or any two of R₁, R₂, R₃, R₄, R₅, R₆, R₇, and R₈ together with the carbon atom to which they are attached form an optionally substituted carbocyclic or heterocyclic fused ring;
A, A′, B, B′ are each independently hydrogen or an optionally substituted group selected from the group comprising hydrogen, alkyl, heteroalkyl, aryl and heteroaryl,
or A and B, or A′ and B′, together with the carbon atom to which they are attached form an optionally substituted carbocyclic or heterocyclic ring;
n is an integer selected from 0 or 1,
wherein when n is 1, X represents a linker connecting both imidate nitrogen atoms via 3 to 8 consecutive bonds; X is an optionally substituted group selected from alkylene, heteroalkylene, arylene, heteroarylene and optionally containing one or more heteroatoms;
or wherein when n is 0, X represents a linker connecting the imidate nitrogen atom via 3 to 8 consecutive bonds to a chelating substituent excluding a hydroxyl, alkoxy, aryloxy, amino substituent; X is a substituted group selected from alkyl, heteroalkyl, aryl, heteroaryl,
or wherein when n is 0 and the chelating substituent is R₁ excluding a methoxy and chlorine substituent; X represents a group selected from an unsubstituted alkyl, heteroalkyl, aryl and heteroaryl;
or wherein when n is 0, X represents an optionally substituted heteroatom comprising nitrogen, oxygen, phosphorous or sulfur with the proviso that the cyclic imidate of formula (I) is chiral.

The invention further provides a process for the preparation of a compound of formula (I), by reacting a compound of formula (II), or a salt thereof, with a reagent of formula X—NH₂ (for n=j) or a reagent of formula H₂N—X—NH₂ (for n=1), wherein R1-R4, R5-R8, A, B and X have the meaning as described above.

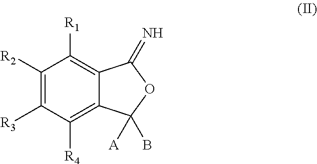

In an embodiment of the process, R1 to R4 equals R5 to R8.

In a preferred embodiment of the process, n=0 and X is selected from a group comprising trans-2-hydroxy-1-indanyl, 1-indanyl, [2-(diphenylphosphino)ferrocen-1-yl]-1-ethyl, 2-[(11b)-3H-Binaphtho[2,1-c:1′,2′-e]phosphepin-4(5H)-yl]ethyl and 2-methoxymethyl-pyrrolidin-1-yl.

In a preferred embodiment of the process, n=1 and X is selected from the group comprising alkyl, trans-1,2-cyclohexadiyl, bis-endo-norbornane-2,5-diyl, or trans-2,2-dimethyl-1,3-dioxolane-4,5-dimethyl or trans-1,2,3,6,7,8-hexahydro-as-indacene-1,8-diyl, aryl and 1,1′-binapht-2,2′-diyl.

In a further aspect, the invention provides a cyclic imidate of formula (I) or a stereoisomeric form thereof or a salt thereof, obtained by a process according to an embodiment of the invention.

In a further aspect, the invention provides a cyclic imidate of formula (I), or a stereoisomeric form thereof or a salt thereof,

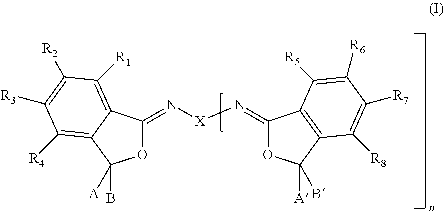

wherein
R₁, R₂, R₃, R₄, R₅, R₆, R₇, and R₈ are each independently selected from the group comprising hydrogen, halogen, alkyl, heteroalkyl, aryl, heteroaryl, hydroxyl, optionally substituted amino, diarylphosphanyl, diheteroarylphosphanyl, arylalkylphosphanyl, heteroarylalkylphosphanyl and dialkylphosphanyl
or any two of R₁, R₂, R₃, R₄, R₅, R₆, R₇, and R₈ together with the carbon atom to which they are attached form an optionally substituted carbocyclic or heterocyclic fused ring;
A, A′, B, B′ are each independently hydrogen or an optionally substituted group selected from the group comprising hydrogen, alkyl, heteroalkyl, aryl and heteroaryl,
or A and B, or A′ and B′, together with the carbon atom to which they are attached form an optionally substituted carbocyclic or heterocyclic ring;
n is 1,
X represents a linker connecting both imidate nitrogen atoms via 3 to 8 consecutive bonds; X is an optionally substituted group selected from alkylene, heteroalkylene, arylene, heteroarylene and optionally containing one or more heteroatoms.

In an embodiment of the above cyclic imidate of the invention, R1, R2, R3, R4 have an identical meaning as R5, R6, R7, R8 and A, B have an identical meaning as A′, B′.

In a preferred embodiment of the above cyclic imidate of the invention, X is selected from the group comprising alkyl, trans-1,2-cyclohexadiyl, bis-endo-norbornane-2,5-diyl, or trans-2,2-dimethyl-1,3-dioxolane-4,5-dimethyl or trans-1,2,3,6,7,8-hexahydro-as-indacene-1,8-diyl, aryl and 1,1′-binapht-2,2′-diyl.

In another aspect, the invention provides a cyclic imidate of formula (I), or a stereoisomeric form thereof or a salt thereof,

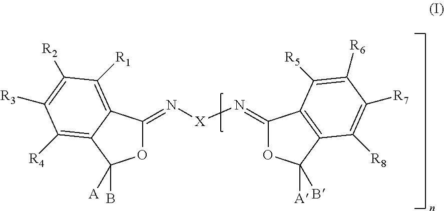

wherein
R₁, R₂, R₃, R₄, R₅, R₆, R₇, and R₈ are each independently selected from the group comprising hydrogen, halogen, alkyl, heteroalkyl, aryl, heteroaryl, hydroxyl, optionally substituted amino, diarylphosphanyl, diheteroarylphosphanyl, arylalkylphosphanyl, heteroarylalkylphosphanyl and dialkylphosphanyl
or any two of R₁, R₂, R₃, R₄, R₅, R₆, R₇, and R₈ together with the carbon atom to which they are attached form an optionally substituted carbocyclic or heterocyclic fused ring;
A, A′, B, B′ are each independently hydrogen or an optionally substituted group selected from the group comprising hydrogen, alkyl, heteroalkyl, aryl and heteroaryl,
or A and B, or A′ and B′, together with the carbon atom to which they are attached form an optionally substituted carbocyclic or heterocyclic ring;
n is 0,
wherein when X represents a linker connecting the imidate nitrogen atom via 3 to 8 consecutive bonds to a chelating substituent excluding a hydroxyl, alkoxy, aryloxy, amino substituent, X is a substituted group selected from alkyl, heteroalkyl, aryl, heteroaryl;
or wherein when the chelating substituent is R₁ excluding a methoxy and chlorine substituent;
X represents a group selected from an unsubstituted alkyl, heteroalkyl, aryl and heteroaryl;
or if X represents an optionally substituted heteroatom comprising nitrogen, oxygen, phosphorous or sulfur then the cyclic imidate of formula (I) is chiral.

In an embodiment of the invention, the cyclic imidate is as described above, that if X represents a linker connecting the imidate nitrogen atom via 3 to 8 consecutive bonds to a chelating substituent, the chelating substituent is not an amide, carboxyl or thiol substituent;

or if X represents an optionally substituted heteroatom comprising nitrogen, oxygen, phosphorous or sulfur, the cyclic imidate of formula (I) is chiral non racemic.

In a preferred embodiment of the cyclic imidate of the invention, wherein R1, R2, R3 and R4 are hydrogen and X is selected from a group comprising trans-2-hydroxy-1-indanyl, 1-indanyl, [2-(diphenylphosphino)ferrocen-1-yl]-1-ethyl, 2-[(11b)-3H-Binaphtho[2,1-c:1′,2′-e]phosphepin-4(5H)-yl]ethyl and 2-methoxymethyl-pyrrolidin-1-yl.

In a preferred embodiment of the cyclic imidate of the invention, the cyclic imidate is a chiral non-racemic compound.

The invention further provides a catalyst, wherein the catalyst is formed by complexing a catalyst precursor comprising a metal and a cyclic imidate containing substructure (Y) as a minimal structural motive, wherein the carbon atoms and the nitrogen atom can be optionally substituted by a chemical substituent.

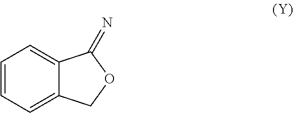

In a preferred embodiment of the catalyst of the invention, the cyclic imidate is a cyclic imidate according to an embodiment of the invention as described above.

The invention further provides in a use of the catalyst according to an embodiment of the invention in the synthesis of chiral non-racemic building blocks for pharmaceuticals, agrochemicals, flavors and/or fragrances.

The invention further provides in a use of the catalyst according to an embodiment of the invention in the synthesis of achiral or racemic building blocks for organic syntheses.

### DETAILED DESCRIPTION

The present invention provides a cyclic imidate of formula (I), or a salt thereof

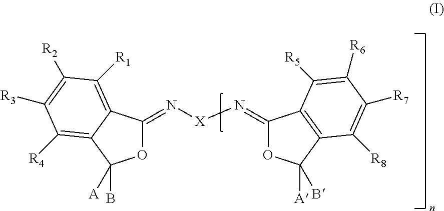

wherein the R1 to R4 and R5 to R8 groups may be the same or different and are, independently of one another, a chemical substituent. This is preferably, but not limited to, a hydrogen atom, a halogen atom, preferably chlorine or bromine, an alkyl or heteroalkyl group, an aryl or heteroaryl group, a hydroxyl group, an optionally substituted amino group or a diarylphosphanyl, diheteroarylphosphanyl, arylalkylphosphanyl, heteroarylalkylphosphanyl or dialkylphosphanyl group group. Any two vicinal R-groups can also, taken together, represent an optionally substituted carbocyclic or heterocyclic fused ring.

A,A′ and B,B′ are independently of one another a hydrogen, an alkyl, a heteroalkyl, an aryl or a heteroaryl group and can be optionally substituted. A and B can also, taken together, represent a ring which can be optionally substituted.

If n is 1, X represents a linker, preferably, but not limited to, an alkyl, heteroalkyl, aryl or heteroaryl group which can be optionally substituted, and which can also contain heteroatoms. The linker is connecting both imidate nitrogen atoms via 3-8 consecutive bonds.

If n is 0, X represents a substituted alkyl, heteroalkyl, aryl or heteroaryl group containing at least one chelating substituent, preferably, but not limited to a diarylphosphanyl, diheteroarylphosphanyl, arylalkylphosphanyl, heteroarylalkylphosphanyl or dialkylphosphanyl group. The chelating substituent is connected to the imidate nitrogen via 3-8 consecutive bonds; the chelating substituent can act together with the imidate nitrogen as a bidentate ligand for a metal.

If n is 0, some prior art exists when X is a substituted alkyl or aryl group containing a hydroxyl, alkoxy or aryloxy (OR), or an amino substituent: these structures were used as synthetic intermediates in the synthesis of organic molecules (Ref. 2). However, these structures have never been used in catalysis as a ligand for a metal.

Alternatively, if n is 0, and R1 is a chelating substituent, preferably, but not limited to a diarylphosphanyl, diheteroarylphosphanyl, arylalkylphosphanyl, heteroarylalkylphosphanyl or dialkylphosphanyl group, or a hydroxyl group, X may also represent an unsubstituted alkyl, heteroalkyl, aryl or heteroaryl group.

If n is 0, and R1 is a chelating group, some prior art exists when R1=OMe, Cl: when R1=Cl, the imidate was used as a dye (Ref. 3) When R1=OMe, these structures were used as synthetic intermediates in the synthesis of organic molecules. (Ref. 4) However these structures have never been used in catalysis as a ligand for a metal.

Alternatively, if n is 0, X may also represent a heteroatom, preferably, but not limited to a substituted nitrogen atom, on the condition that the thus obtained cyclic imidate is chiral.

Preferably, the cyclic imidates (I) as described above are chiral and non-racemic, however not excluding achiral and racemic cyclic imidates.

Preferably in a cyclic imidate (I) as described above, n is 1 and R1 to R4 equals R5 to R8.

More preferably, in a cyclic imidate (I) as described above with n is 1 and R1 to R4 equals R5 to R8, R1 to R8 are hydrogen or halogen atoms such as chlorine or bromine or combinations thereof.

Most preferably, in a cyclic imidate (I) as described above with n is 1 and R1 equals R2, R1 and R2 are hydrogen or halogen atoms such as chlorine or bromine, X is selected from a group comprising an alkyl or aryl group, preferably trans-1,2-cyclohexadiyl, 1,1′-binapht-2,2′-diyl, bis-endo-norbornane-2,5-diyl, trans-1,2,3,6,7,8-hexahydro-as-indacene-1,8-diyl, or trans-2,2-dimethyl-1,3-dioxolane-4,5-dimethyl.

Alternatively, in a cyclic imidate (I) as described above, when n is 0, preferably R1 is hydrogen and X is selected from a group comprising trans-2-hydroxy-1-indanyl, 1-indanyl, (Rp)-2-(diphenylphosphino)ferrocenyl-1-(1S)-1-ethyl, 2-[(11bS)-3H-binaphtho[2,1-c:1′,2′-e]phosphepin-4(5H)-yl]ethyl or 2-methoxymethyl-pyrrolidin-1-yl.

The present invention provides a catalyst, wherein the catalyst is formed by complexing a catalyst precursor comprising a metal, and a cyclic imidate containing substructure (Y) as a minimal structural motive, wherein the carbon atoms and the nitrogen atom can be optionally substituted by any chemical substituent.

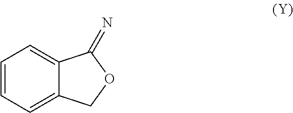

The present invention provides the use of a cyclic imidate as a ligand for catalysis purposes in which the ligand contains substructure (Y) as a minimal structural motive, wherein the carbon atoms and the nitrogen atom can be optionally substituted by any chemical substituent.

The present invention provides a process for the preparation of a compound of formula (I), by reacting a compound of formula (II), or a salt thereof, with a reagent of formula X—NH₂ (for n=0) or a reagent of formula H₂N—X—NH₂ (for n=1).

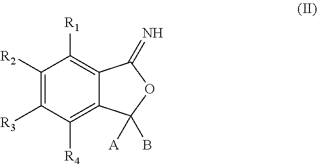

The cyclic imidates of the present invention are stable. They are accessible through commercially available or readily obtainable starting materials in high yield via an efficient one-step synthesis starting from imidate (II) (FIG. 1) and a primary amine (for n=0) or diamine (for n=1). The synthesis is modular: a set of imidates (II) can be combined with a set of primary amines, resulting in an imidate ligand family. This is important because most of the time ligands have to be “tailored” to a substrate. The process can be scaled up to produce industrial quantities. Chiral non-racemic cyclic imidates are obtained upon using a chiral non-racemic amine or diamine, or a chiral non-racemic cyclic imidate precursor (II), or a combination of both.

In a first aspect, we introduced imidates (I) as a new class of ligands. Preferably, the cyclic imidates (I) are chiral non-racemic ligands suitable for application in asymmetric synthesis.

In a second aspect, the present invention provides a catalyst, wherein the catalyst is formed by complexing a catalyst precursor comprising a metal, with a cyclic imidate (I) as described above.

In a third aspect, the catalysts according to the invention are particularly useful for asymmetric syntheses such as, but not limited to, aziridinations, diethylzinc-additions, cyclopropanations and allylic alkylations. Reactions resulted in high yields. Enantioselectivity can be tuned via variation of R1 to R8, X or A,A′ and B,B′ in the catalyst.

In a fourth aspect, the present invention provides the use of a catalyst as described above in the synthesis of chiral building blocks for e.g. pharmaceuticals, agrochemicals, flavors and/or fragrances. However, this does not exclude their use as catalysts for the synthesis of achiral building blocks.

### I. Imidate Ligands

The present invention provides novel imidates. These imidates are compounds of formula (I), or salts thereof, wherein the R1 to R4 and R5 to R8 groups may be the same or different and are, independently of one another a chemical substituent. This is preferably, but not limited to, a hydrogen atom, a halogen atom, preferably chlorine or bromine, an alkyl or heteroalkyl group, an aryl or heteroaryl group, a hydroxyl group, an optionally substituted amino group or a diarylphosphanyl, diheteroarylphosphanyl, arylalkylphosphanyl, heteroarylalkylphosphanyl or dialkylphosphanyl group group.

Any two vicinal R-groups can also, taken together, represent an optionally substituted carbocyclic or heterocyclic fused ring.

The R₁- to R₈-groups may be the same or different and are, independently of one another, hydrogen atoms, optionally substituted hydrocarbon groups having from 1 to 16 carbon atoms, halogen (F, CI, Br, I), phosphino (PRR′), amino (NRR′), imino (—N═CRR′), hydrazino (NR—NR′R″), hydroxyl (OH), alkoxy (OR), sulfhydryl (SH), alkylthio (SR), phosphine oxide (P(═O)RR′), phosphinato (P(═O)ORR′, OP(═O)RR′), phosphonato (P(═O)OROR′, OP(═O)ORR′), phosphate (OP(═O)OROR′), phosphinito (OPRR′), phosphonito (OPORR′), phosphito (OP(OR)₂), aminophosphino (R″N—PRR′), phosphoramidite (R″N—P(OR)₂), iminophosphino (N═PRR′R″), nitrile (CN), alkoxycarbonyl (COOR), nitro (NO₂) and sulfonyl (SO₃H). In these formulas, R, R′ and R″ are, independently of one another, (optionally substituted) alkyl, cycloalkyl, hetero-cycloalkyl, aryl, heteroaryl. Any two R-groups from R, R′ and R″ can also, taken together, represent a ring (cycloalkyl or hetero-cycloalkyl).

A,A′ and B,B′ are independently of one another a hydrogen, an alkyl, a heteroalkyl, an aryl or a heteroaryl group and can be optionally substituted. A and B can also, taken together, represent a ring which can be optionally substituted.

If n is 1, X represents a linker, preferably, but not limited to, an alkyl, heteroalkyl, aryl, heteroaryl group which can be optionally substituted, and which can also contain heteroatoms. The linker is connecting both imidate nitrogen atoms via 3-8 consecutive bonds. In case of symmetrical bidentate imidates, more preferably R1 equals R5, R2 equals R6, R3 equals R7 and R4 equals R8; A equals A′ and B equals B′.

If n is 0, X represents a substituted alkyl, heteroalkyl, aryl or heteroaryl group containing at least one chelating substituent, preferably, but not limited to a diarylphosphanyl, diheteroarylphosphanyl, arylalkylphosphanyl, heteroarylalkylphosphanyl or dialkylphosphanyl group group. The chelating substituent is connected to the imidate nitrogen via 3-8 consecutive bonds; the chelating substituent can act together with the imidate nitrogen as a bidentate ligand for a metal.

If n=0, X represents a substituted alkyl, heteroalkyl, cycloalkyl, hetero-cycloalkyl, aryl or heteroaryl group containing at least one chelating substituent (e.g. phosphino (PRR′), amino (NRR′), imino (═NR or —N═CRR′), hydrazino (NR—NR′R″ or NR—N═CR′R″ or ═N—NRR′), hydroxylamino (NR—OR′ or O—NRR′ or ═N—OR), imidato (N═C(R)OR′), amidino (N═C(R)NR′R″), hydroxyl (OH), alkoxy (OR), sulfhydryl (SH), alkylthio (SR), phosphine oxide (P(═O)RR′), phosphinato (P(═O)ORR′, OP(═O)RR′), phosphonato (P(═O)OROR′, OP(═O)ORR′), phosphate (OP(═O)OROR′), phosphinito (OPRR′), phosphonito (OPORR′), phosphito (OP(OR)₂), aminophosphino (R″N—PRR′), phosphoramidite (R″N—P(OR)₂), iminophosphino (N═PRR′R″), halogen (F, Cl, Br, I), connected to the imidate nitrogen via 3 to 6 consecutive bonds, or a chelating substituent (imidato (C(═NR)OR′), amidino (C(═N)NRR′)) connected to the imidate nitrogen via 2 to 6 consecutive bonds, and which can act, together with the imidate nitrogen, as a bidentate ligand for a metal. In these formulas, R, R′ and R″ are, independently of one another, (optionally substituted) alkyl, cycloalkyl, hetero-cycloalkyl, aryl, heteroaryl. Any two R-groups can also, taken together, represent a ring (cycloalkyl or hetero-cycloalkyl.

If n is 0, some prior art exists when X is a substituted alkyl or aryl group containing a hydroxyl, alkoxy or aryloxy (OR), or an amino substituent: these structures were used as synthetic intermediates in the synthesis of organic molecules (Ref. 2). However, these structures have never been used in catalysis as a ligand for a metal.

Alternatively, if n is 0, and R1 is a chelating substituent, preferably, but not limited to an optionally substituted amino group, a diarylphosphanyl, diheteroarylphosphanyl, arylalkylphosphanyl, heteroarylalkylphosphanyl or dialkylphosphanyl group, or a hydroxyl group, X may also represent an unsubstituted alkyl, heteroalkyl, aryl or heteroaryl group.

If n=0, and R₁ represents a chelating substituent (e.g. phosphino (PRR′), amino (NRR′), imino (—N═CRR′), imidato (N═C(R)OR′ or C(═NR)OR′), hydrazino (NR—NR′R″ or NR—N═CR′R″), hydroxylamino (NR—OR′ or O—NRR′), hydroxyl (OH), alkoxy (OR), sulfhydryl (SH), alkylthio (SR), phosphine oxide (P(═O)RR′), phosphinato (P(═O)ORR′, OP(═O)RR′), phosphonato (P(═O)OROR′, OP(═O)ORR′), phosphate (OP(═O)OROR′), phosphinito (OPRR′), phosphonito (OPORR′), phosphito (OP(OR)₂), aminophosphino (R″N—PRR′), phosphoramidite (R″N—P(OR)₂), iminophosphino (N═PRR′R″), halogen (F, Cl, Br, I). In these formulas, R, R′ and R″ are, independently of one another, (optionally substituted) alkyl, cycloalkyl, hetero-cycloalkyl, aryl, heteroaryl. Any two R-groups can also, taken together, represent a ring (cycloalkyl or hetero-cycloalkyl.

If n is 0, and R1 is a chelating group, some prior art exists when R1=OMe, Cl: when R1=Cl, the imidate was used as a dye. (Ref. 3). When R1=OMe, these structures were used as synthetic intermediates in the synthesis of organic molecules (Ref. 4). However these structures have never been used in catalysis as a ligand for a metal.

Alternatively, if n is 0, X may also represent a heteroatom, preferably, but not limited to a substituted nitrogen atom, on the condition that the thus obtained cyclic imidate is chiral. X can alternatively represent an amino (NRR′), alkoxy (OR), phosphino (PRR′) or phosphinito (P(OR′)₂) group, with R, R′ are, independently of one another, (optionally substituted) alkyl, cycloalkyl, hetero-cycloalkyl, aryl, heteroaryl; R and R′ can also, taken together, represent a ring (cycloalkyl or hetero-cycloalkyl).

“halogen atom” refers to fluorine, chlorine, iodine or bromine. The preferred halogen is chlorine or bromine.

“alkyl” refers to a substituted or unsubstituted, straight, branched or cyclic hydrocarbon chain containing from 1 to 15 carbon atoms. Preferred alkyl groups are lower alkyl groups, i.e. alkyl groups containing from 1 to 6 carbon atoms. Preferred cycloalkyls have from 3 to 10 carbon atoms, preferably 3-6 carbon atoms in their ring structure. Suitable examples of unsubstituted alkyl groups include methyl, ethyl, propyl, isopropyl, cyclopropyl, butyl, iso-butyl, tert-butyl, sec-butyl, cyclobutyl, pentyl, cyclopentyl, hexyl, cyclohexyl, and the like.

“heteroalkyl” refers to an alkyl group containing one or more heteroatoms in the chain.

“aryl” refers to any aromatic carbocyclic group. The aryl group can be monocyclic (e.g. phenyl) or polycyclic (e.g. naphthyl) and can be unsubstituted or substituted.

“heteroaryl” refers to an aryl group containing one or more heteroatoms (e.g. 2-furyl, 2-pyridyl).

“salts” refers to hydrochloride, hydrobromide or hydrosulfate salts.

“chelating substituent” refers to a chemical substituent comprising a heteroatom with a lone pair capable of forming a coordinative bond, such as O, P, N, S or a halogen.

Chelating substituents are especially important on position R1 where they can act, together with the imidate nitrogen, as a bidentate ligand for a metal. On other places than R1, substituents are especially important to modulate the electron density of the imidate.

The cyclic imidates according to the invention are obtainable via a process comprising the steps of: reacting a compound of formula (II), or a salt thereof, with a primary amine of formula XNH₂ (for n=0) or a diamine of formula H₂N—X—NH₂ (for n=1) wherein X has the meaning as set forth above.

In a preferred embodiment R1 to R4 equals R5 to R8 in a cyclic imidate of formula (I). In a more preferred embodiment, n is 1 and R1 to R4 equals R5 to R8 in a cyclic imidate of formula (I). In a most preferred embodiment, R1 to R8 are hydrogen or halogen in a cyclic imidate of formula (I). Preferably the halogen is chlorine or bromine.

A compound of formula (II), or a salt thereof, is obtainable via a process comprising transformation of an ortho-cyanoarylaldehyde of formula (III) into the compound of formula (II) (FIG. 1).

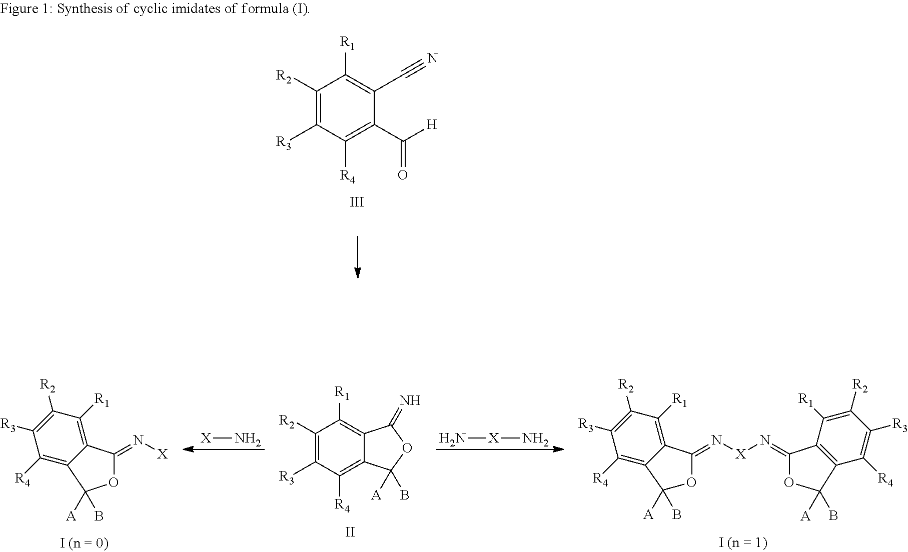

Ortho-cyano-benzaldehyde III-A is commercially available. Substituted ortho-cyanoarylaldehydes of formula (III) are obtainable from commercially available substituted 2-methylbenzonitriles (VI), as depicted in FIG. 2.

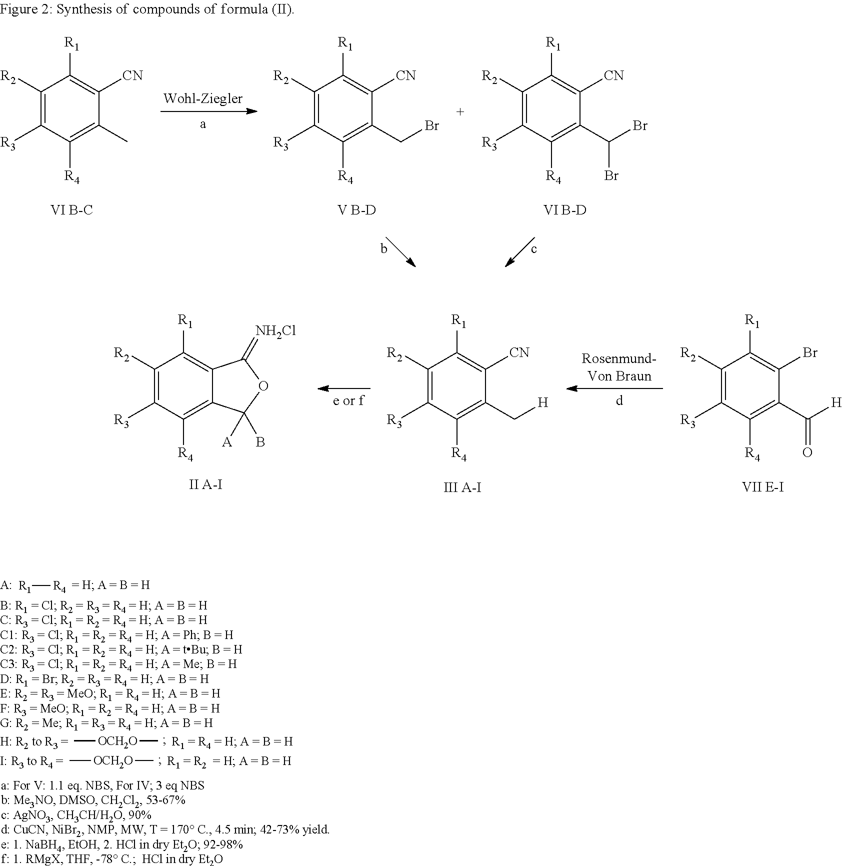

A Wohl-Ziegler reaction with 1.1 equivalents NBS (N-bromosuccinimide) delivered the desired monobromide V. However, formation of a certain amount of dibromide IV could not be prevented. This resulted in lower yields of compounds of formula (V) and a difficult separation. Moreover, low yields were also obtained in the oxidation of the monobromine V with Me₃NO. The inventors found that reaction of VI with 3 equivalents of NBS resulted selectively in the dibrominated product IV in excellent yield. Hydrolysis of IV with AgNO₃ in CH₃CN/H₂O delivered III in very high yields.

A second possibility to access these substituted ortho-formylbenzonitriles (III) is via a Rosenmund-Von Braun reaction (step d in FIG. 2). This reaction was performed under microwave irradiation in less than five minutes.

Compounds of formula II, were obtained from treatment of 2-cyanobenzaldehydes (III B-C) with NaBH₄ in ethanol. The compounds of formula II, were isolated as a hydrochloric acid salt in high yield (92-96%).

Preferred methods for the synthesis of compounds of formula III, in particular 2-cyanobenzaldehydes (III B-C), from compounds V or IV are as follows:

2-Chloro-6-(bromomethyl)benzonitrile (V-B). A solution of 2-chloro-6-methylbenzonitrile VI-B (4.83 g, 31.9 mmol), NBS (6.24 g, 35.1 mmol) and benzoylperoxide (232.0 mg, 0.96 mmol) in CCl₄ (100 mL) was refluxed for 7 h. Afterwards, the solids are filtered off and the filtrate was concentrated in vacuo. The crude product was purified by flash chromatography over silica gel (pentane/Et₂O, 90/10) resulting in pure V-B, 4.35 g (82%). No formation of the dibromo product IV-B was observed. ¹H NMR (300 MHz, CDCl₃): δ 4.60 (s, 2H), 7.43-7.54 (m, 3H). ¹³C NMR (75.4 MHz, CDCl₃): δ 28.9 (CH₂), 113.4 (C), 113.9 (C), 128.5 (CH), 129.7 (CH), 133.7 (CH), 137.7 (C), 143.4 (C). IR(HATR): 3070, 3025, 2227, 1588, 1567, 1455, 1443, 1264, 1219, 1203, 1180, 1155, 1117, 988, 905, 796, 780, 737, 628, 609 cm⁻¹. EI-MS m/z (rel intensity %): 231 (M⁺, 10), 229 (M⁺, 8), 152 (33), 150 (100), 123 (27), 114 (22), 81 (18), 79 (18), 63 (21), 50 (14). Melting point: 83° C.

2-(Bromomethyl)-4-chlorobenzonitrile (V-C). The reaction was performed on 4-chloro-2-methylbenzonitrile VI-C (2.0 g, 13.2 mmol) according to the typical procedure for V-B. The crude product was purified by flash chromatography over silica gel (pentane/Et₂O, 96/4) resulting in pure V-C, 1.74 g (57%). Formation of the dibromo product IV-C was also observed, 0.97 g (24%). For V-C: ¹H NMR (300 MHz, CDCl₃): δ 4.57 (s, 2H), 7.39 (dd, J=2.0, 8.3 Hz, 1H), 7.55 (d, J=2.0 Hz, 1H), 7.59 (d, J=8.3 Hz, 1H). ¹³C NMR (75.4 MHz, CDCl₃): δ 28.2 (CH₂), 110.8 (C), 116.0 (C), 129.4 (CH), 130.8 (CH), 134.2 (CH), 139.7 (C), 142.8 (C). IR (HATR): 3080, 3035, 2224, 1592, 1564, 1480, 1438, 1404, 1284, 1230, 1222, 1180, 1105, 1080, 900, 882, 827, 742, 726, 630, 618 cm⁻¹. EI-MS m/z (rel intensity %): 233 (M⁺, 25), 231 (M⁺,100), 229 (M⁺, 77), 203 (9), 152 (6), 150 (18), 114 (66), 87 (31), 63 (35). Melting point: 78° C.

2-Bromo-6-(bromomethyl)benzonitrile (V-D). The reaction was performed on 2-bromo-6-methylbenzonitrile VI-D (1.0 g, 5.1 mmol) according to the typical procedure for V-B. The crude product was purified by flash chromatography over silicagel (hexane/EtOAc, 95/5) resulting in pure IV-D, 825.0 mg (46%). Formation of the monobrominated product V-D was also observed, 616.7 mg (44%). For V-D: ¹H-NMR (300 MHz, CDCl₃): δ 6.98 (s, 1H), 7.54 (J=7.9 Hz, 1H), 7.66 (d, J=7.9 Hz, 1H), 7.99 (d, J=7.9 Hz, 1H) ppm. ¹³C-NMR (75.4 MHz, CDCl₃): δ 35.2 (CH), 111.8 (C), 114.3 (C), 125.3 (C), 128.6 (CH), 133.9 (CH), 134.3 (CH), 146.7 (C) ppm. IR (HATR): 3072, 3010, 2228, 1586, 1557, 1449, 1434, 1319, 1289, 1244, 1233, 1198, 1174, 1144, 1118, 868, 792, 732, 648 cm⁻¹. EI-MS m/z (rel. intensity %): 355 (M⁺, <5), 353 (M⁺, <5), 274 (100), 114 (62), 88 (25), 63 (25). Melting Point: 116° C. HRMS (EI): calcd for C₈H₄⁷⁹Br₃N, 350.7894; found 350.7886. For IV-D: ¹H-NMR (300 MHz, CDCl₃): δ 4.62 (s, 2H), 7.43 (t, J=7.8 Hz, 1H), 7.49 (d, J=7.8 Hz, 1H), 7.63 (d, J=7.8 Hz, 1H) ppm. ¹³C-NMR (75.4 MHz, CDCl₃): δ 29.2 (CH₂), 115.1 (C), 115.8 (C), 126.3 (C), 129.0 (CH), 132.9 (CH), 133.8 (CH), 143.7 (C) ppm. IR (HATR): 3079, 3066, 3029, 2977, 2953, 2925, 2872, 2232, 1718, 1581, 1559, 1446, 1438, 1312, 1285, 1260, 1222, 1201, 1177, 1150, 1110, 894, 857, 798, 767, 739, 621 cm⁻¹. EI-MS m/z (rel. intensity %): 275 (M⁺, 9), 196 (98), 194 (100), 115 (52), 88 (24), 79 (15), 62 (22), 49 (18). Melting Point: 126° C. HRMS (EI): calcd for C₈H₅⁷⁹Br₂N, 272.8789; found 272.8778.

2-Chloro-6-(dibromomethyl)benzonitrile (IV-B). A solution of 2-chloro-6-methylbenzonitrile VI-B (9.91 g, 65.4 mmol), NBS (35.22 g, 197.9 mmol) and benzoylperoxide (534.0 mg, 2.2 mmol) in CCl₄ (100 mL) was refluxed overnight. Afterwards, the solids are filtered off and the filtrate was concentrated in vacuo. The crude product was purified by flash chromatography over silica gel (hexane/EtOAc, 95/5) resulting in pure IV-B, 19.04 g (94%). ¹H-NMR (300 MHz, CDCl₃): δ 6.96 (s, 1H), 7.49 (dd, J=0.8, 8.1 Hz, 1H), 7.62 (t, J=8.1 Hz, 1H), 7.94 (dd, J=0.8, 8.1 Hz, 1H) ppm. ¹³C-NMR (75.4 MHz, CDCl₃): δ 35.0 (CH), 109.5 (C), 113.1 (C), 128.0 (CH), 130.6 (CH), 134.1 (CH), 136.9 (C), 146.3 (C) ppm. IR (HATR): 3076, 3008, 2232, 1589, 1567, 1454, 1439, 1292, 1251, 1238, 1174, 1140, 1134, 891, 796, 779, 735, 651, 633 cm⁻¹. EI-MS m/z (rel intensity %): 309 (M⁺, 2), 232 (25), 230 (100), 228 (74), 149 (14), 114 (39), 87 (17), 74 (9), 63 (16), 50 (13). Melting point: 120° C.

4-Chloro-2-(dibromomethyl)-benzonitrile (IV-C). The reaction was performed on 4-chloro-2-methylbenzonitrile (9.80 g, 64.6 mmol) according to the typical procedure for IV-B. The crude product was purified by flash chromatography over silicagel (hexane/EtOAc, 95/5) resulting in pure IV-C, 19.55 g (98%). ¹H-NMR (300 MHz, CDCl₃): δ 6.92 (s, 1H), 7.41 (dd, J=2.0, 8.4 Hz, 1H), 7.55 (d, J=8.4 Hz, 1H), 8.00 (d, J=2.0, 1H) ppm. ¹³C-NMR (75.4 MHz, CDCl₃): δ 34.3 (CH), 106.9 (C), 115.2 (C), 130.4 (CH), 130.6 (CH), 133.5 (CH), 140.5 (C), 145.9 (C) ppm. IR (HATR): 3080, 3058, 3028, 3004, 2359, 2227, 1589, 1556, 1481, 1462, 1404, 1304, 1279, 1206, 1170, 1138, 1114, 1081, 902, 820, 742, 689, 649, 622 cm⁻¹. EI-MS m/z (rel intensity %): 309 (M⁺, 2), 232 (25), 230 (100), 228 (73), 149 (16), 114 (47), 87 (20), 74 (10), 63 (20), 50 (15). Melting point: 120° C.

2-Chloro-6-formylbenzonitrile (III-B). To a solution of IV-B (18.0 g, 58.2 mmol) in CH₃CN (60 mL) was added a solution of AgNO₃ (39.5 g, 23.3 mmol) in H₂O (32 mL). The resulting yellow suspension was refluxed during 20 min. The solids were filtered off and washed with CH₂Cl₂ (150 mL). The combined filtrate was washed with H₂O (25 mL), dried over Na₂SO₄ and concentrated in vacuo. The crude product was purified by flash chromatography over silica gel (hexane/EtOAc, 2/1) resulting in pure III-B, 8.67 g (90%). ¹H-NMR (300 MHz, CDCl₃): δ 7.72 (t, J=7.9 Hz, 1H), 7.79 (dd, J=1.2, 7.9 Hz, 1H), 7.94 (dd, J=1.2, 7.9 Hz, 1H), 10.31 (s, 1H) ppm. ¹³C-NMR (75.4 MHz, CDCl₃): δ 112.9 (C), 114.4 (C), 127.5 (CH), 133.8 (CH), 134.9 (CH), 138.5 (C), 139.0 (C), 187.6 (CH) ppm. IR (HATR): 3069, 2865, 2221, 1697, 1584, 1566, 1443, 1395, 1291, 1224, 1195, 1182, 1155, 922, 798, 781, 726, 675 cm⁻¹. EI-MS m/z (rel intensity %): 167 (5); 165 (15), 139 (33), 137 (100), 110 (24), 101 (44), 84 (13), 75 (79), 61 (23), 50 (45). Melting point: 140° C.

4-Chloro-2-formyl-benzonitrile (III-C). The reaction was performed on IV-C (18.07 g, 58.4 mmol) according to the typical procedure for III-B. The crude product was purified by flash chromatography over silicagel (hexane/EtOAc, 9/1) resulting in pure III-C, 8.04 g (83%). ¹H-NMR (300 MHz, CDCl₃): δ 7.71 (dd, J=2.0, 8.3 Hz, 1H), 7.77 (d, J=8.3 Hz, 1H), 8.00 (d, J=2.0 Hz, 1H), 10.31 (s, 1H) ppm. ¹³C-NMR (75.4 MHz, C₆D₆): δ 111.9 (C), 115.2 (C), 129.2 (CH), 133.3 (CH), 134.6 (CH), 138.0 (C), 139.3 (C), 186.2 (CH) ppm. IR (HATR): 3101, 3069, 2871, 2226, 1698, 1584, 1558, 1485, 1376, 1294, 1203, 1119, 1099, 897, 839, 744, 702, 620 cm⁻¹. EI-MS m/z (rel intensity %): 167 (10); 165 (29), 139 (33), 137 (100), 110 (26), 102 (44), 100 (33), 75 (55), 61 (16), 50 (39). Melting point: 119° C.

2-Bromo-6-formylbenzonitrile (III-D). The reaction was performed on IV-D (1.35 g, 3.8 mmol) according to the typical procedure for III-B. The crude product was purified by flash chromatography over silicagel (hexane/EtOAc, 8/2) resulting in pure III-D, 603.0 mg (76%). ¹H-NMR (300 MHz, CDCl₃): δ 7.64 (t, J=7.9 Hz, 1H), 7.95 (d, J=7.9 Hz, 1H), 7.98 (d, J=7.9 Hz, 1H), 10.29 (s, 1H) ppm. ¹³C-NMR (75.4 MHz, CDCl₃): δ 114.1 (C), 116.9 (C), 127.6 (C), 127.9 (CH), 133.9 (CH), 138.0 (CH), 187.7 (CH) ppm. IR (HATR): 3078, 2921, 2854, 1698, 1641, 1579, 1557, 1436, 1390, 1279, 1219, 1176, 1134, 892, 847, 786, 722, 666 cm⁻¹. EI-MS m/z (rel. intensity %): 211 (M⁺, 12), 209 (M⁺, 13), 183 (93), 181 (94), 102 (100), 84 (11), 75 (93), 61 (12), 50 (53). Melting point: 124° C. HRMS (EI): calcd for C₈H₄⁷⁹BrNO: 208.9476; found 208.9474.

4,5-Dimethoxy-2-formylbenzonitrile (III-E). 2-Bromo-5-methoxybenzaldehyde VII-E (2.50 g, 10.0 mmol), CuCN (5.48 g, 61.2 mmol) and NiBr₂ (892 mg, 4.1 mmol) were dissolved in 50 mL NMP. The reaction mixture was irradiated in a microwave oven for 4.5 min (T=170° C., pₘₐₓ=17 bar, 200 W, powermax on). Next, the reaction mixture was poured into H₂O (600 mL) and extracted with CH₂Cl₂ (3×600 mL). The combined organic phases were dried on MgSO₄, evaporated in vacuo and purified by flash chromatography over silicagel (Hexane/EtOAc, 70/30) resulting in pure III-E, 1.41 g (73%). ¹H-NMR (300 MHz, CDCl₃): δ 3.99 (s, 1H), 7.16 (s, 1H), 7.47 (s, 1H), 10.25 (s, 1H) ppm. ¹H-NMR (75.4 MHz, CDCl₃): δ 56.4 (CH₃), 56.7 (CH₃), 108.2 (C), 109.4 (CH), 114.4 (CH), 116.0 (C), 131.8 (C), 152.9 (C), 153.7 (C), 187.5 (CH) ppm. IR (HATR): 3060, 2855, 2220, 1684, 1584, 1512, 1474, 1458, 1440, 1401, 1357, 1289, 1262, 1224, 1201, 1092, 988, 882, 753, 733, 634 cm⁻¹. EI-MS: 191 (M⁺).

2-Formyl-4-methoxy-benzonitrile (III-F). 2-Bromo-5-methoxybenzaldehyde VII-F (2.5 g, 11.6 mmol), CuCN (6.25 g, 69.8 mmol) and NiBr₂ (838.0 mg, 3.84 mmol) were dissolved in 50 mL NMP. The reaction mixture was irradiated in a microwave oven for 4.5 min (T=170° C., pₘₐₓ=17 bar, 200 W, powermax on). Next, the reaction mixture was poured into H₂O (600 mL) and extracted with CH₂Cl₂ (3×600 mL). The combined organic phases were dried on MgSO₄, evaporated in vacuo and purified by flash chromatography over silicagel (Hexane/EtOAc, 70/30) resulting in pure III-F, 738.0 mg (42%). ¹H-NMR (300 MHz, CDCl₃): δ 3.93 (s, 3H), 7.21 (dd, J=2.8, 8.7 Hz, 1H), 7.50 (d, J=2.8 Hz, 1H), 7.73 (d, J=8.7 Hz, 1H), 10.3 (s, 1H) ppm. ¹³C-NMR (75.4 MHz, CDCl₃): δ 56.0 (CH₃), 106.0 (C), 112.9 (CH), 116.2 (C), 120.9 (CH), 135.4 (CH), 138.8 (C), 163.1 (C), 188.4 (CH) ppm. IR (HATR): 2920, 2223, 1688, 1600, 1565, 1490, 1460, 1281, 1254, 1191, 1116, 1026, 919, 896, 829, 772, 681 cm⁻¹. EI-MS: 161 (M⁺).

2-Formyl-5-methyl-benzonitrile (III-G). 2-Bromo-4-methylbenzaldehyde VII-G (2.5 g, 12.3 mmol), CuCN (5.52 g, 61.6 mmol) and NiBr₂ (807.0 mg, 3.69 mmol) were dissolved in 50 mL NMP. The reaction mixture was irradiated in a microwave oven for 4.5 min (T=170° C., pₘₐₓ=17 bar, 200 W, powermax on). Next, the reaction mixture was poured into H₂O (600 mL) and extracted with CH₂Cl₂ (3×600 mL). The combined organic phases were dried on MgSO₄, evaporated in vacuo and purified by flash chromatography over silicagel (Hexane/EtOAc, 70/30) resulting in pure III-G, 790.6 mg (45%). ¹H-NMR (300 MHz, CDCl₃): δ 2.48 (s, 3H), 7.56 (d, J=8.0 Hz, 1H), 7.62 (s, 1H), 7.93 (d, J=8.0 Hz, 1H), 10.28 (s, 1H) ppm. ¹³C-NMR (75.4 MHz, CDCl₃): δ 21.5 (CH₃), 113.9 (C), 116.1 (C), 129.6 (CH), 133.9 (CH), 134.4 (CH), 134.6 (C), 145.8 (C), 188.3 (CH) ppm. IR (HATR): 3194, 2222, 1697, 1597, 1573, 1452, 1390, 1309, 1211, 1156, 1116, 1045, 835, 805 cm⁻¹. EI-MS: 145 (M⁺).

6-Cyano-1,3-benzodioxol-5-carboxaldehyde (III-H). 6-Bromo-1,3-benzodioxol-5-carboxaldehyde VII-H (2.5 g, 10.9 mmol), CuCN (5.87 g, 65.5 mmol) and NiBr₂ (954.0 mg, 4.37 mmol) were dissolved in 50 mL NMP. The reaction mixture was irradiated in a microwave oven for 4.5 min (T=170° C., pₘₐₓ=17 bar, 200 W, powermax on). Next, the reaction mixture was poured into H₂O (600 mL) and extracted with CH₂Cl₂ (3×600 mL). The combined organic phases were dried on MgSO₄, evaporated in vacuo and purified by flash chromatography over silicagel (Hexane/EtOAc, 70/30) resulting in pure III-H, 717.4 mg (38%). ¹H-NMR (300 MHz, CDCl₃): δ 6.19 (s, 2H), 7.14 (s, 1H), 7.43 (s, 1H) 10.22 (s, 1H) ppm. ¹³C-NMR (75.4 MHz, CDCl₃): δ 103.5 (CH₂), 107.6 (CH), 110.2 (C), 112.2 (CH), 115.7 (C), 134.2 (C), 152.1 (C), 152.5 (C), 186.9 (CH) ppm. IR (HATR): 2917, 2847, 2232, 1682, 1594, 1504, 1487, 1434, 1367, 1286, 1049, 1029, 924, 900, 789 cm⁻¹. EI-MS: 175 (M⁺).

5-Cyano-1,3-benzodioxol-4-carboxaldehyde (III-I). 5-Bromo-1,3-benzodioxol-4-carboxaldehyde VII-I (2.0 g, 8.7 mmol), CuCN (4.70 g, 52.4 mmol) and NiBr₂ (763.2 mg, 3.50 mmol) were dissolved in 40 mL NMP. The reaction was irradiated in a microwave oven for 4.5 min (T=170° C., pₘₐₓ=17 bar, 200 W, powermax on). Next, the reaction mixture was poured into H₂O (600 mL) and extracted with CH₂Cl₂ (3×600 mL). The combined organic phases were dried on MgSO₄, evaporated in vacuo and purified by flash chromatography over silicagel (Hexane/EtOAc, 70/30) resulting in pure III-I, 561.0 mg (38%). ¹H-NMR (300 MHz, CDCl₃): δ 6.26 (s, 2H), 7.04 (d, J=8.0 Hz, 1H), 7.35 (d, J=8.0 Hz, 1H), 10.30 (s, 1H) ppm. ¹³C-NMR (75.4 MHz, CDCl₃): δ 104.1 (CH₂), 112.4 (CH), 116.3 (C), 126.0 (C), 130.1 (CH), 152.8 (C), 185.6 (CH) ppm. EI-MS: 175 (M⁺).

Cyclic imidates of formula (I) are preferably prepared from compounds of the formula (II) as follows:

1,3-Dihydro-iminoisobenzofuran hydrochloride (II-A). 2-Formylbenzonitrile (7.0 g, 53.4 mmol) was dissolved in absolute ethanol (420 mL) and cooled to −78° C. NaBH₄ was added and the reaction mixture was allowed to heat to 0° C. in 30 min. The reaction mixture was poured into H₂O and extracted with CH₂Cl₂ (3×1000 mL). The organic phases were dried over Na₂SO₄ and concentrated in vacuo. The resulting orange oil was dissolved in CH₂Cl₂ (165 mL) and dry HCl in Et₂O (65 mL) was added. The resulting suspension was filtrated and the white crystals were washed with dry THF. This resulted in 8.3 g (92%) of imidate (1). ¹H-NMR (300 MHz, CD₃OD) δ 5.99 (s, 2H), 7.76 (t, J=7.8 Hz, 1H), 7.84 (d, J=7.8 Hz, 1H), 7.98 (t, J=7.8 Hz, 1H), 8.33 (d, J=7.8 Hz, 1H). ¹³C-NMR (75.4 MHz, CD₃OD): δ 81.0 (CH₂), 123.9 (CH), 124.6 (C), 126.5 (CH), 131.1 (CH), 138.1 (CH), 148.9 (C), 178.4 (C). IR (HATR): 3422, 3357, 3062, 3036, 2924, 2806, 2717, 2628, 1676, 1617, 1592, 1560, 1486, 1446, 1330, 1318, 1222, 1080, 938, 794, 739 cm⁻¹. EI-MS m/z (rel. intensity %): 133 ((M⁺-HCl), 50), 104 (100), 89 (15), 77 (44), 63 (14), 51 (20), 43 (7). ES-MS: 134 [M-Cl⁻]⁺. Melting point: decomposition. HRMS (EI) calculated for C₈H₇ON 133.0528; found 133.0533.

7-Chloro-1,3-dihydro-iminoisobenzofuran hydrochloride (II-B). The reaction as described for II-A was performed on 2-chloro-6-formylbenzonitrile (III-B) (2.0 g, 12.1 mmol) according to the typical procedure resulting in 2.32 g (94%) of imidate ester hydrochloride (II-B). ¹H-NMR (300 MHz, DMSO-d₆) δ 5.93 (s, 2H), 7.78 (d, J=7.8 Hz, 1H), 7.80 (d, J=7.8 Hz, 1H), 7.94 (t, J=7.8 Hz, 1H) ppm. ¹³C-NMR (75.4 MHz, DMSO-d₆): δ 78.0 (CH₂), 121.1 (C), 121.8 (CH), 130.3 (CH), 130.5 (C), 137.9 (CH), 150.3 (C), 173.6 (C) ppm. IR (HATR): 3053, 2936, 2861, 2706, 2628, 2545, 2436, 1662, 1610, 1582, 1524, 1474, 1430, 1408, 1322, 1306, 1228, 1196, 1156, 1132, 1060, 1042, 919, 857, 792, 763, 727, 654 cm⁻¹. EI-MS m/z (rel. intensity %): 169 (M⁺, 11), 167 (M⁺, 33), 140 (33), 138 (100), 111 (10), 102 (47), 89 (74), 75 (69), 63 (42), 50 (50), 43 (19). ES-MS: 168 [m-Cl⁻]⁺. Mp: decomposition.

5-Chloro-1,3-dihydro-iminoisobenzofuran hydrochloride (II-C). The reaction as described for II-A was performed on 2-formyl-4-chlorobenzonitrile (III-C) (2.0 g, 12.1 mmol) according to the typical procedure, resulting in 2.38 g (96%) of imidate ester hydrochloride (II-C). ¹H-NMR (300 MHz, CD₃OD) δ 5.94 (s, 2H), 7.79 (dd, J=0.9, 8.5 Hz, 1H), 7.88 (d, J=0.9 Hz, 1H), 8.20 (d, J=8.5 Hz, 1H) ppm. ¹³C-NMR (75.4 MHz, CD₃OD): δ 80.5 (CH₂), 123.7 (C), 124.4 (CH), 127.8 (CH), 131.8 (CH), 144.7 (C), 150.7 (C), 177.6 (C) ppm. IR (HATR): 2801, 1671, 1643, 1613, 1586, 1545, 1464, 1447, 1417, 1310, 1290, 1212, 1173, 1119, 1082, 1067, 943, 894, 864, 855, 834, 790, 774, 752, 660 cm⁻¹. EI-MS m/z (rel. intensity %): 169 (M⁺, 16), 167 (M⁺, 48), 140 (33), 138 (100), 132 (20), 111 (20), 102 (44), 89 (21), 75 (60), 63 (36), 50 (86), 43 (21). ES-MS: 168 [M-Cl⁻]⁺. Mp: decomposition.

7-Bromo-1,3-dihydro-iminoisobenzofuran hydrochloride (II-D). The reaction was performed on 2-bromo-6-formylbenzonitrile (III-D) (500.0 mg, 2.4 mmol) according to the typical procedure described for II-A, resulting in 403.1 mg (69%) of imidate ester hydrochloride (II-D). ¹H-NMR (300 MHz, DMSO-d₆): δ 5.90 (s, 2H), 7.84-7.88 (m, 2H), 7.94-7.99 (m, 1H) ppm. ¹³C-NMR (75.4 MHz, DMSO-d₆): δ 77.6 (CH₂), 118.6 (C), 122.3 (CH), 122.6 (C), 133.8 (CH), 137.8 (CH), 150.6 (C), 174.3 (C) ppm. IR (HATR): 3328, 3154, 2670, 1682, 1602, 1577, 1514, 1467, 1444, 1404, 1319, 1295, 1226, 1191, 1150, 1124, 1058, 1046, 934, 895, 794, 745, 724, 660 cm⁻¹. EI-MS m/z (rel. intensity %): 213 ([M-Cl]⁺, 71), 211 ([M-Cl]⁺, 66), 184 (98), 182 (100), 157 (13), 132 (9), 102 (60), 89 (36), 75 (67), 63 (52), 51 (55). Mp: decomposition. HRMS (EI): calcd for C₈H₇⁷⁹Br³⁵ClNO: 246.9400; found 246.9386.

5,6-Dimethoxy-1,3-dihydro-iminoisobenzofuran hydrochloride (II-E). The reaction was performed on 4,5-dimethoxy-2-formylbenzonitrile (III-E) (1.0 g, 5.2 mmol) according to the typical procedure described for II-A, resulting in 1.18 g (99%) of imidate ester hydrochloride (II-E). ¹H-NMR (300 MHz, DMSO-d₆): δ 3.84 (s, 3H), 3.93 (s, 3H), 5.84 (s, 2H), 7.39 (s, 1H), 8.34 (s, 1H) ppm. ¹³C-NMR (75.4 MHz, DMSO-d₆): 56.1 (CH₃), 56.5 (CH₃), 78.6 (CH₂), 104.4 (CH), 106.7 (CH), 114.7 (C), 143.0 (C), 150.1 (C), 156.4 (C), 175.3 (C) ppm. IR (HATR): 2838, 1703, 1608, 1591, 1503, 1485, 1453, 1406, 1365, 1307, 1296, 1275, 1230, 1101, 1059, 1018, 978, 941, 866, 784 cm⁻¹. ES-MS: 194 [M-Cl⁻]⁺. Mp: decomposition.

5-Methoxy-1,3-dihydro-iminoisobenzofuran hydrochloride (II-F). The reaction was performed on 2-formyl-4-methoxy-benzonitrile (III-F) (0.500 g, 3.1 mmol) according to the typical procedure described for II-A, resulting in 501.4 mg (81%) of imidate ester hydrochloride (II-F). ¹H-NMR (300 MHz, DMSO-d₆): δ 3.42 (s, 1H), 3.92 (s, 3H), 5.87 (s, 2H), 7.29 (d, J=8.8 Hz, 1H), 7.35 (s, 1H), 8.54 (d, J=8.8 Hz, 1H) ppm. ¹³C-NMR (75.4 MHz, DMSO-d₆): δ 56.4 (CH₃), 78.4 (CH₂), 106.5 (CH), 115.4 (C), 117.6 (CH), 127.7 (CH), 150.7 (C), 166.0 (C), 174.7 (C) ppm. IR (HATR): 2847, 1718, 1590, 1491, 1445, 1422, 1312, 1274, 1245, 1110, 1066, 1014, 932, 914, 861, 816, 784, 678 cm⁻¹. ES-MS: 164 [M-Cl⁻]⁺. Mp: decomposition.

6-Methyl-1,3-dihydro-iminoisobenzofuran hydrochloride (II-G). The reaction was performed on 2-formyl-5-methyl-benzonitrile (III-G) (1.0 g, 6.9 mmol) according to the typical procedure described for II-A, resulting in 1.079 g (86%) of imidate ester hydrochloride (II-G). ¹H-NMR (300 MHz, DMSO-d₆): δ 2.42 (s, 3H), 5.90 (s, 2H), 7.69 (d, J=8.0 Hz, 1H), 7.75 (d, J=8.0 Hz, 1H), 8.52 (s, 1H) ppm. ¹³C-NMR (75.4 MHz, DMSO-d₆): δ 20.8 (CH₃), 79.1 (CH₂), 122.4 (CH), 123.8 (C), 125.7 (CH), 137.3 (CH), 139.3 (C), 144.6 (C), 175.3 (C) ppm. IR (HATR): 2828, 1707, 1614, 1500, 1456, 1402, 1332, 1301, 1232, 1111, 1070, 944, 807, 753 cm⁻¹. ES-MS: 148 [M-Cl⁻]⁺. Mp: decomposition.

5,6-Methylenedioxy-1,3-dihydro-iminoisobenzofuran (II-H). The reaction was performed on 6-cyano-1,3-benzodioxol-5-carboxaldehyde (III-H) (1.0 g, 5.71 mmol) according to the typical procedure described for II-A, resulting in 1.00 g (99%) of imidate ester (II-H). ¹H-NMR (300 MHz, DMSO-d₆): δ 5.13 (s, 2H), 6.04 (s, 2H), 6.72 (s, 1H), 7.17 (s, 1H). ¹³C-NMR (75.4 MHz, DMSO-d₆): δ 71.4 (CH₂), 101.4 (CH), 102.2 (CH₂), 103.2 (CH), 139.4 (C), 148.8 (C), 152.0 (C) ppm. IR (HATR): 3284, 2914, 1676, 1498, 1471, 1459, 1365, 1278, 1260, 1136, 1038, 1003, 960, 934, 872, 812, 742 cm⁻¹. ES-MS: 178 [M-Cl⁻]⁺. Mp: decomposition.

4,5-Methylenedioxy-1,3-dihydro-iminoisobenzofuran (II-I). The reaction was performed on 5-cyano-1,3-benzodioxol-4-carboxaldehyde (III-I) (0.5 g, 2.85 mmol) according to the typical procedure described for II-A, resulting in 499.0 mg (99%) of imidate ester (II-I). ES-MS: 178 [M-Cl⁻]⁺.

5-Chloro-3-phenyl-1,3-dihydro-iminoisobenzofuran hydrochloride (II-C1). 2-Formyl-4-chlorobenzonitrile (III-C) (0.1 g, 0.604 mmol) was dissolved in dry THF (6 mL) and cooled to −78° C. PhMgBr (3M in Et₂O, 0.919 mmol, 0.306 mL) was added and the mixture was allowed to react for 2.5 h at −78° C. The reaction mixture was poured into H₂O (20 mL) and extracted with CH₂Cl₂ (3×20 mL). The organic phases were dried over Na₂SO₄ and concentrated in vacuo. The resulting yellow oil was dissolved in CH₂Cl₂ (2.4 mL) and a saturated solution of dry HCl in Et₂O (1 mL) was added. The resulting suspension was filtrated and the white crystals were washed with dry THF (2 mL). This resulted in 96.4 mg (57%) of imidate hydrochloride (II-C1). ¹H-NMR (300 MHz, DMSO-d₆) δ 7.30 (s, 1H), 7.43-7.46 (m, 4H), 7.70 (s, 1H), 7.89 (dd, J=1.2, 8.2 Hz, 1H), 8.88 (d, J=8.6 Hz, 1H) ppm. ¹³C-NMR (75.4 MHz, DMSO-d₆): δ 91.2 (CH), 123.1 (C), 123.4 (CH), 127.9 (CH), 128.3 (CH), 129.1 (CH), 130.3 (CH), 130.7 (CH), 133.4 (C), 141.9 (C), 150.6 (C), 173.5 (C) ppm. ES-MS: 244 [M-Cl⁻]⁺.

5-Chloro-3-t.butyl-1,3-dihydro-iminoisobenzofuran hydrochloride (II-C2). 2-Formyl-4-chlorobenzonitrile (III-C) (0.1 g, 0.604 mmol) was dissolved in dry THF (6 mL) and cooled to −78° C. t.BuMgBr (1M in THF, 0.604 mmol, 0.604 mL) was added and the mixture was allowed to react for 2 h at −78° C. The reaction mixture was poured into H₂O (20 mL) and extracted with CH₂Cl₂ (3×20 mL). The organic phases were dried over Na₂SO₄ and concentrated in vacuo. The resulting yellow solid was dissolved in CH₂Cl₂ (2.4 mL) and a saturated solution of dry HCl in Et₂O (1 mL) was added. The resulting suspension was filtrated and the white crystals were washed with dry THF (2 mL). This resulted in 16.2 mg (10%) of imidate (II-C2). ¹H-NMR (300 MHz, DMSO-d₆) δ 0.97 (s, 9H), 6.02 (s, 1H), 7.86-7.89 (m, 2H), 8.89 (d, J=8.2, 1H) ppm. ¹³C-NMR (75.4 MHz, DMSO-d₆): δ 24.8 (CH₃), 35.6 (C), 97.1 (CH), 123.5 (C), 123.9 (CH), 128.4 (CH), 130.6 (CH), 141.6 (C), 149.0 (C), 173.1 (C) ppm. ES-MS: 224 [M-Cl⁻]⁺.

5-Chloro-3-methyl-1,3-dihydro-iminoisobenzofuran hydrochloride (II-C3). 2-Formyl-4-chlorobenzonitrile (III-C) (0.1 g, 0.604 mmol) was dissolved in dry THF (6 mL) and cooled to −78° C. MeMgCl (3M in THF, 0.604 mmol, 0.201 mL) was added and the mixture was allowed to react for 2.5 h at −78° C. The reaction mixture was poured into H₂O (20 mL) and extracted with CH₂Cl₂ (3×20 mL). The organic phases were dried over Na₂SO₄ and concentrated in vacuo. The resulting yellow solid was dissolved in CH₂Cl₂ (2.4 mL) and a saturated solution of dry HCl in Et₂O (1 mL) was added. The resulting suspension was filtrated and the white crystals were washed with dry THF (2 mL). This resulted in 51.3 mg (39%) of imidate (II-C3). ¹H-NMR (300 MHz, DMSO-d₆) δ 1.69 (d, J=6.8, 3H), 6.26 (q, J=6.4, 6.8, 1H), 7.86-7.89 (m, 2H), 7.81 (d, J=8.3, 1H), 8.00 (s, 1H), 8.74 (d, J=8.3, 1H) ppm. ¹³C-NMR (75.4 MHz, DMSO-d₆): δ 18.6 (CH₃), 88.1 (CH), 122.7 (C), 122.8 (CH), 128.0 (CH), 130.2 (CH), 141.5 (C), 152.6 (C), 173.4 (C) ppm. ES-MS: 182 [M-Cl⁻]⁺.

All reactions were carried out under argon atmosphere in dry solvents under anhydrous conditions, unless otherwise stated. Benzaldehyde was passed through basic alumina. All other reagents were purchased and used without purification, unless otherwise noted. Flash chromatography was carried out with Rocc silica gel (0.040-0.063 mm). ¹H-NMR, ¹³C-NMR were recorded on a Bruker Avance 300 or a Bruker AM 500 spectrometer as indicated, with chemical shifts reported in ppm relative to TMS, using the residual solvent signal as a reference. IR-spectra were recorded on a Perkin-Elmer spectrum 1000 FT-IR spectrometer with a Pike Miracle HATR module. El-Mass spectra were recorded with a Hewlett-Packard 5988A mass spectrometer. ES-Mass spectra were recorded with an Agilent 1100 series single quadrupole MS detector type VL with an API-ES source. Analytical chiral HPLC-separations were performed on an Agilent 1100 series HPLC system with DAD detection. Exact molecular masses were measured on a Kratos MS50TC mass spectrometer.

I.1. Imidate Ligands from Amines

The cyclic imidates according to the invention are obtainable via a process comprising the steps of: reacting a compound of formula (II), or a salt thereof, with a primary amine of formula XNH₂ (for n=0) or a diamine of formula H₂N—X—NH₂ (for n=1) wherein X has the meaning as set forth above.

In a preferred embodiment, a cyclic imidate of formula (I) is obtained via a process comprising the steps of: reacting a compound of formula (II), or a salt thereof, with a primary diamine of formula H₂N—X—NH₂ (for n=1) selected from a group of compounds comprising (1R,2R)-(−)-diaminocyclohexane, (R)-(+)-1,1′-binaphthyl-2,2′-diamine, (1S,2S,4S,5S)-2,5-diamino-norbornane, (4S,5S)-4,5-di(aminomethyl)-2,2-dimethyldioxolane, (1R,8R)-1,2,3,6,7,8-hexahydro-as-indacene-1,8-diamine, (1R,2R)-(−)-trans-1-amino-2-indanol, (R)-(−)-aminoindane.

Preferred embodiments of cyclic imidates of formula (I) are compounds wherein n is 1, R1 to R4 equals R5 to R8, R1 to R8 are hydrogen, halogen or a combination thereof and X is a linker, comprising an alkyl or aryl group, preferably trans-1,2-cyclohexadiyl, 1,1′-binapht-2,2′-diyl, bis-endo-norbornane-2,5-diyl, trans-1,2,3,6,7,8-hexahydro-as-indacene-1,8-diyl, or trans-2,2-dimethyl-1,3-dioxolane-4,5-dimethyl as depicted in Table 1.

TABLE 1
Preferred bidentate imidate compounds of formula (I)

| Ligand | R1 to R8 |
|---|---|
| 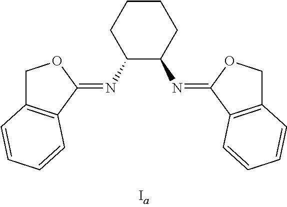 | R1 to R8 = H |
| 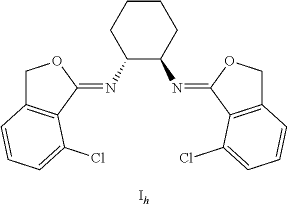 | R1 = R5 = Cl R2 = R3 = R4 = R6 = R7 = R8 = H |
| 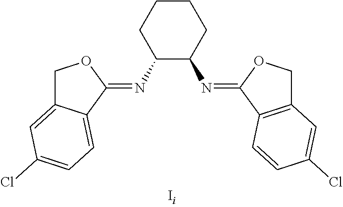 | R3 = R7 = Cl R1 = R2 = R4 = R5 = R6 = R8 = H |
| 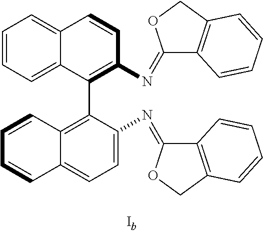 | R1 to R8 = H |
| 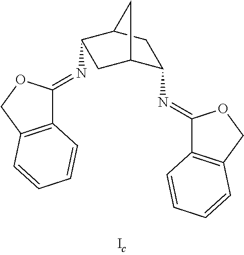 | R1 to R8 = H |
| 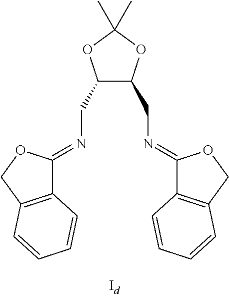 | R1 to R8 = H |
| 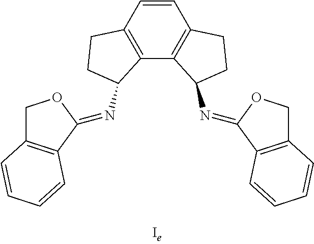 | R1 to R8 = H |

In another preferred embodiment, a compound of formula (I) is obtained from a compound of formula (II), or a salt thereof, by reaction with a primary amine of formula XNH₂ (for n=0).

Preferred embodiments of cyclic imidates of formula (I) are cyclic imidates wherein n is 0, R₁ equals R₂, R₁ and R₂ are hydrogen or halogen and X is an alkyl group, preferably trans-2-hydroxy-1-indanyl, 1-indanyl, (Rp)-2-(diphenylphosphino)ferrocenyl-1-(1S)-1 ethyl, 2-[(11bS)-3H-Binaphtho[2,1-c:1′,2′-e]phosphepin-4(5H)-yl]ethyl or 2-methoxymethyl-pyrrolidin-1-yl, as depicted in Table 2.

TABLE 2
Preferred monodentate imidate compounds of formula (II)

| Ligand | R1 to R4 |
|---|---|
| 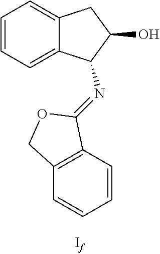 | R1 to R4 = H |
| 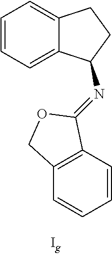 | R1 to R4 = H |
| 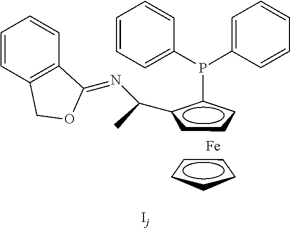 | R1 to R4 = H |
| 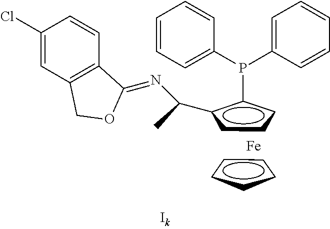 | R1 = R2 = R4 = H R3 = Cl |
| 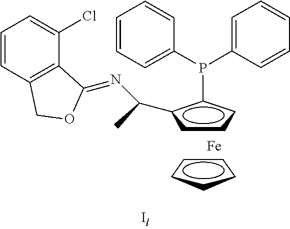 | R1 = Cl R2 = R4 = R3 = H |
| 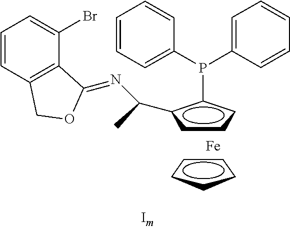 | R1 = Br R2 = R4 = R3 = H |
| 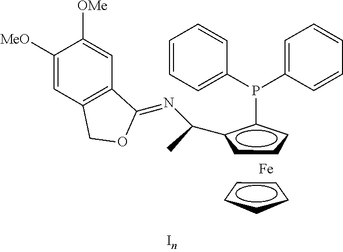 | R1 = R4 = H R2 = R3 = MeO |
| 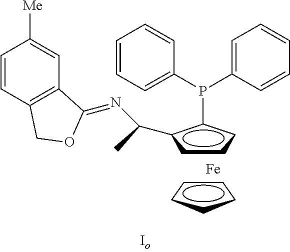 | R1 = R3 = R4 = H R2 = Cl |
| 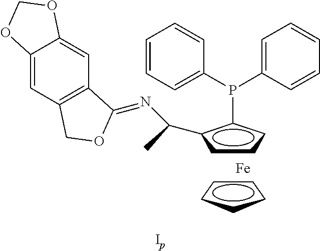 | R1 = R4 = H R2 = R3 = —OCH₂O— |
| 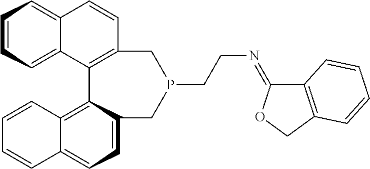 | R1 to R4 = H |
| 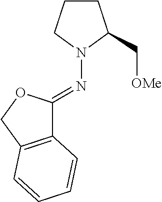 | R1 to R4 = H |

In a preferred embodiment, the cyclic imidates of formula (I) are chiral. In a preferred embodiment, chiral imidates of formula (I) are obtained from chiral amines. Preferably the chiral amines are as depicted below (2a to 2h). Use of 2f or 2g will provide a monodentate ligand, whereas use of 2a to 2e will provide a bidentate ligand. Use of 2h will provide a mixed phosphine-imidate ligand.

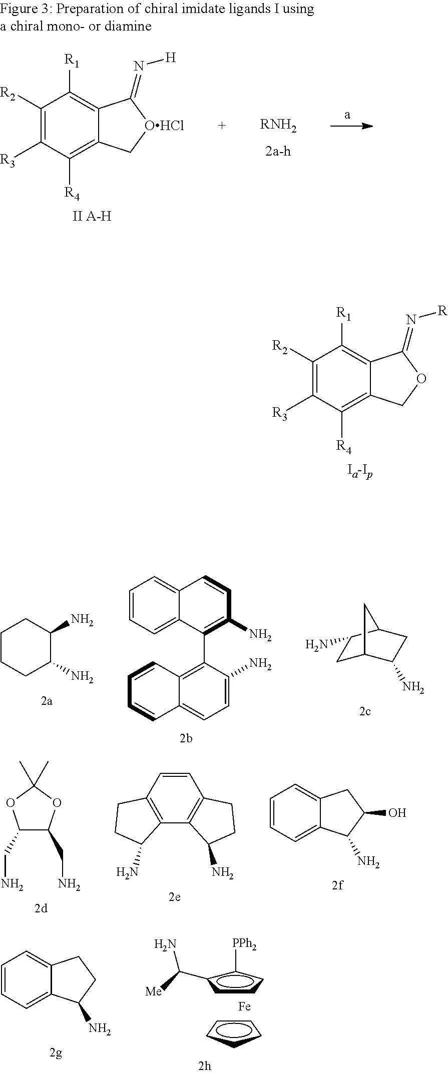

Chiral compounds of formula (I), in particular chiral imidate esters, may be obtained from the amines 2a to 2h as described below. Results obtained are summarized in Table 3.

N,N-bis-(3H-isobenzofuran-1-ylidene)-cyclohexane-(1R,2R)-diamine (Iₐ).

A suspension of (1R,2R)-(−)-diaminocyclohexane (119 mg, 1.04 mmol) and imidate 1′-A (450 mg, 2.65 mmol) in dry CH₂Cl₂ (5 mL) was cooled in an ice bath. Et₃N (1 mL, 13.6 mmol) was added and the resulting suspension was refluxed for 24 h. The reaction mixture was passed through a short pad of silica gel and eluted with EtOAc. Evaporation in vacuo and purification by flash chromatography over silica gel (toluene/Et₂O, 6/4, +1% Et₃N) resulted in (Iₐ) as a white solid, 308 mg (85%). ¹H-NMR (500 MHz, CDCl₃): δ 1.47 (m, 2H) 1.60 (m, J=1.9, 10.7 Hz, 2H), 1.80 (m, J=1.9 Hz, 2H), 1.92 (m, J=3.9, 10.7 Hz, 2H), 3.98 (dd, J=3.9, 4.7 Hz, 2H), 5.16 (d, J=14.3 Hz, 2H), 5.23 (d, J=14.3 Hz, 2H), 7.23 (td, J=0.7, 7.5 Hz, 2H), 7.3 (dt, J=0.7, 7.5 Hz, 2H), 7.38 (dt, J=0.9, 7.5 Hz, 2H), 7.76 (d, J=7.5 Hz, 2H) ppm. ¹³C-NMR (75.4 MHz, CDCl₃): 24.9 (CH₂), 32.5 (CH₂), 61.0 (CH), 71.7 (CH₂), 121.1 (CH), 123.4 (CH), 128.0 (CH), 130.6 (CH), 130.9 (C), 143.1 (C), 158.9 (C) ppm. IR (HATR): 3040, 2927, 2873, 2854, 1689, 1614, 1468, 1448, 1360, 1288, 1227, 1093, 1015, 951, 863, 775, 726, 702, 670 cm⁻¹. EI-MS m/z (rel. intensity %): 346 (M⁺, 22), 213 (22), 186 (10), 160 (30), 146 (20), 118 (70), 104 (46), 90 (100), 63 (15), 41 (12). ES-MS: 347 [M+H]⁺. [α]_{D}²⁰=+84.8 (c 1.12, CHCl₃). Mp: 146° C. HRMS (EI) calculated for C₂₂H₂₂O₂N₂ 346.1681; found 346.1680.

(R)-(+)-N,N′-bis-(3H-isobenzofuran-1-ylidene)-1,1′-binaphthyl-2,2′-diamine (I_{b}). A suspension of (R)-(+)-1,1′-binaphthyl-2,2′-diamine (99 mg, 0.35 mmol) and imidate 1′-A (179 mg, 1.06 mmol) in MeOH (5 mL) was cooled in an ice bath. Et₃N (0.32 mL, 2.3 mmol) was added and the resulting suspension was refluxed for 5 days. Evaporation in vacuo and purification by flash chromatography over silica gel (toluene/EtOAc, 7/3, +1% Et₃N) resulted in I_{b} as a white solid, 127.8 mg (71%). ¹H-NMR (300 MHz, CDCl₃) δ 4.07 (d, J=14.6 Hz, 2H), 4.84 (d, J=14.6 Hz, 2H), 7.10-7.56 (m, 16H), 7.85 (m, 4H). ¹³C-NMR (75.4 MHz, CDCl₃): 71.8 (CH₂), 120.8 (CH), 122.9 (CH), 123.9 (CH), 124.6 (CH), 125.6 (CH), 126.5 (C), 126.9 (CH), 127.5 (CH), 127.6 (CH), 128.4 (CH), 130.5 (C), 130.7, 131.4 (CH), 133.8 (C), 143.2 (C), 144.3 (C), 158.3 (C). IR (HATR): 3050, 2357, 1687, 1614, 1589, 1502, 1466, 1361, 1291, 1262, 1206, 1408, 1004, 942, 826, 770, 727 cm⁻¹. EI-MS m/z (rel. intensity %): 516 (M⁺, 16), 382 (29), 284 (12), 266 (18), 149 (32), 118 (31), 90 (83), 45 (100). ES-MS: 517 [M+H]⁺. [α]_{D}²⁰=+596.6 (c 1.01, CHCl₃). Mp: 216-218° C. HRMS (EI) calculated for C₃₆H₂₄O₂N₂ 516.1838; found 516.1837.

(1S,2S,4S,5S)-bis-(3H-isobenzofuran-1-ylidene)-bicyclo[2.2.1]heptane-2,5-diamine (Iₛ). A suspension of (1S,2S,4S,5S)-2,5-diamino-norbornane (410 mg, 3.26 mmol) and imidate II (1.60 mg, 9.46 mmol) in dry CH₂Cl₂ (30 mL) was cooled in an ice bath. Et₃N (2.5 mL, 18 mmol) was added and the resulting suspension was stirred for 16 h at room temperature. Evaporation in vacuo and purification by flash chromatography over silica gel (toluene/Et₂O, 6/4, 1% Et₃N) resulted in white solid. This contained 90% of I_{c} and 10% of endo-exo bisimidate. Recrystallization in CH₂Cl₂/hexane afforded Ic as a pure product, 654.3 mg (56%). ¹H-NMR (500 MHz, CDCl₃) δ 1.59 (s, 2H), 1.93 (m, 2H), 1.95 (m, 2H), 2.38 (s, 2H), 4.18 (m, 2H), 5.29 (s, 4H), 7.31 (d, J=7.6 Hz, 2H), 7.35 (t, J=7.6 Hz, 2H), 7.44 (t, J=7.6 Hz, 2H), 7.87 (d, J=7.6 Hz, 2H) ppm. ¹³C-NMR (75.4 MHz, CDCl₃): 29.8 (CH₂), 38.1 (CH₂), 43.2 (CH), 58.2 (CH), 72.1 (CH₂), 121.1 (CH), 123.8 (CH), 128.5 (CH), 130.6 (C), 131.0 (CH), 143.0 (C), 160.0 (C) ppm. IR (HATR): 3023, 2963, 2860, 2368, 2324, 1679, 1468, 1447, 1362, 1337, 1286, 1062, 1042, 1002, 936, 850, 780, 723 cm⁻¹. EI-MS m/z (rel. intensity %): 358 (M⁺, 9), 317 (9), 239 (9), 225 (24), 198 (24), 184 (32), 159 (23), 134 (27), 118 (69), 90 (100). ES-MS: 359 [M+H]⁺. [α]_{D}²⁰=−54.6 (c 1.24, CHCl₃). Mp: 108° C. HRMS (EI) calculated for C₂₃H₂₂O₂N₂ 358.1681; found 358.1682.

(4S,5S)-4,5-Di(3H-isobenzofuran-1-ylideneamino-methyl)-2,2-dimethyl-1,3-dioxolane (I_{d}). A suspension of (4S,5S)-4,5-di(aminomethyl)-2,2-dimethyl-1,3-dioxolane (105.0 mg, 0.66 mmol) and imidate II-A (307 mg, 1.81 mmol) in CH₂Cl₂ was cooled in an ice bath. Et₃N (0.48 mL, 3.4 mmol) was added and the reaction mixture was stirred for 16 h at room temperature. Evaporation in vacuo and recrystallization from EtOAc resulted in I_{d} as a white solid, 236.6 mg (92%). ¹H-NMR (300 MHz, CDCl₃): δ 1.49 (s, 6H), 3.81 (m, 4H), 4.24 (t, J=3.5 Hz, 2H), 5.25 (s, 4H),), 7.31 (d, J=7.5 Hz, 2H), 7.37 (t, J=7.5 Hz, 2H), 7.46 (dt, J=1.0, 7.5 Hz, 2H), 7.83 (d, J=7.5 Hz, 2H) ppm. ¹³C-NMR (75.4 MHz, CDCl₃): 27.3 (CH₃), 49.8 (CH₂), 72.1 (CH₂), 79.8 (CH), 109.1 (C), 121.2 (CH), 123.7 (CH), 128.3 (CH), 130.4 (C), 131.1 (CH), 143.2 (C), 160.7 (C) ppm. IR (HATR): 2903, 1692, 1367, 1293, 1251, 1166, 1073, 998, 724, 664 cm⁻¹. EI-MS m/z (rel. intensity %): 392 (M⁺, <1), 377 (2), 260 (5), 246 (17), 201 (29), 188 (46), 160 (17), 146 (100), 118 (28), 91 (58). ES-MS: 393 [M+H]⁺. [α]_{D}²⁰=−47.0 (c 1.00, CHCl₃). Mp: 204° C. HRMS (EI) calculated for C₂₃H₂₄O₄N₂ 392.1736; found 392.1737.

(1R,8R)—N,N′-Bis-(3H-isobenzofuran-1-ylidene)-1,2,3,6,7,8-hexahydro-as-indacene-1,8-diamine (Iₑ). (1S,8S)-1,2,3,6,7,8-Hexahydro-as-indacene-1,8-diol (1.5 g, 7.9 mmol), obtainable as described in Ref. 5, was dissolved in dry toluene (60 mL) and cooled to 0° C. Diphenylphosphorazidate (5.2 mL, 24.0 mmol) was added dropwise followed by DBU (3.6 mL, 24.1 mmol). The resulting reaction mixture was stirred for 75 min at 0° C. and for 18 h at room temperature. Water (100 mL) was added to the reaction mixture, which was extracted with toluene (2×100 mL). The organic phase was washed with 0.5N HCl (2×100 mL). Drying with Na₂SO₄, filtration and removal of the volatiles resulted in a crude mixture which was purified by chromatography. The apolar products were separated from the polar products via silica gel chromatography (pentane/ether, 99/1). A second purification via chromatography (pentane/toluene, 90/10) resulted in pure (1R,8R)-1,8-diazido-1,2,3,6,7,8-hexahydro-as-indacene, 1.44 g (76%, >99% ee). ¹H-NMR (500 MHz, CDCl₃): 2.22 (dddd, J=4.4, 5.2, 8.5, 13.6 Hz, 2H), 2.54 (dddd, J=6.5, 7.4, 8.5, 13.6 Hz, 2H), 2.89 (ddd, J=5.2, 8.5, 15.3 Hz, 2H), 3.08 (ddd, J=6.5, 8.5, 15.3 Hz, 2H), 7.20 (s, 2H) ppm. ¹³C-NMR (125.7 MHz, CDCl₃): 30.3 (CH₂), 32.6 (CH₂), 64.8 (CH), 125.5 (CH), 137.0 (C), 142.9 (C) ppm. IR (KBr, thin film): 2942, 2850, 2096, 1467, 1445, 1324, 1245, 1051, 1005, 862, 814 cm⁻¹. EI-MS m/z (rel. intensity %): 240 (M⁺, 2), 198 ([M-N₃]⁺, 100), 169 (25), 155 (27), 128 (25), 115 (36), 63 (33), 51 (25). [α]_{D}²⁰=−256.2 (c 0.69, CHCl₃). Conditions for chiral HPLC analysis: Chiralcel OD-H column, solvent: n-hexane/EtOH (99.5/0.5), flow rate=1 mL/min, T=35° C., retention times: 10-12 min for (1S,8S) and 16-18 min for (1R,8R).

(1R,8R)-1,8-Diazido-1,2,3,6,7,8-hexahydro-as-indacene (1.37 g, 5.71 mmol) was dissolved in toluene (14 mL) and THF (20 mL). PPh₃ (3.78 g, 14.4 mmol) was added and after 2 h of stirring at room temperature, H₂O (10 mL) was added, and stirring was continued overnight. The organic solvents were removed in vacuo and the H₂O phase was extracted with CH₂Cl₂ (150 mL). The organic phase was subsequently extracted with HCl (10%, 2×160 mL). The combined H₂O phases were washed with CH₂Cl₂ (3×350 mL) and evaporated. Dissolving in H₂O (50 mL) and lyophilization resulted in pure (1R,8R)-1,2,3,6,7,8-hexahydro-as-indacene-1,8-diamine hydrochloride (Iₑ) as a white powder, 1.18 g (79%). ¹H-NMR (500 MHz, CD₃OD): 2.28 (dddd, J=1.5, 1.5, 7.5, 14.5 Hz, 2H), 2.58 (dddd, J=7.5, 8.8, 9.3, 14.5 Hz, 2H), 3.02 (ddd, J=1.5, 9.3, 16.6 Hz, 2H), 3.25 (ddd, J=7.5, 8.8, 16.6 Hz, 2H), 5.22 (dd, J=1.5, 7.5 Hz, 2H), 7.43 (s, 2H) ppm. ¹³C-NMR (125.7 MHz, CD₃OD): 30.5 (CH₂), 32.2 (CH₂), 55.4 (CH), 128.5 (CH), 136.5 (C), 146.2 (C) ppm. IR (KBr, thin film): 3422, 3179, 3036, 2096, 2924, 2677, 2580, 1596, 1508, 1474, 1450, 1347, 813 cm⁻¹. ES-MS: 189 [M−(2× HCl)+H]⁺, 172 [M−(2× HCl)−NH₃+H]⁺, 155 [M−(2× HCl)−(2×NH₃)+H]⁺. [α]_{D}²⁰=−106.0 (c 1.05, H₂O). Mp: decomposition.

The HCl salt of as-indacenediamine Iₑ (10.9 mg, 0.0417 mmol) and imidate II-A (20.3 mg, 0.1197) were suspended in dry CH₂Cl₂ (0.75 mL) and cooled in an ice bath. Et₃N (32 μL, 0.230 mmol) was added and the resulting suspension was stirred for 24 h at room temperature. The reaction mixture was passed through a short pad of silica gel and eluted with EtOAc. Evaporation in vacuo and purification by flash chromatography over silica gel (cyclohexane/EtOAc, 2/1) resulted in I, as a white solid, 16.3 mg (93%). ¹H-NMR (300 MHz, C₆D₆) δ 2.20 (dddd, J=8.0, 8.5, 8.5, 12.1 Hz, 2H), 2.64 (dddd, J=2.2, 8.0, 8.5, 12.1 Hz, 2H), 2.86 (ddd, J=8.5, 8.5, 15.0 Hz, 2H), 3.01 (ddd, J=2.2, 8.5, 15.0 Hz, 2H), 3.44 (d, J=14.2 Hz, 2H), 4.28 (d, J=14.2 Hz, 2H), 6.23 (t, J=8.0 Hz, 2H), 6.37-6.45 (m, 2H), 6.85-6.98 (m, 4H), 7.18 (s, 2H), 7.94-8.01 (m, 2H) ppm. ¹³C-NMR (75.4 MHz, C₆D₆): 31.4 (CH₂), 34.7 (CH₂), 60.8 (CH), 71.0 (CH₂), 120.7 (CH), 123.5 (CH), 123.9 (CH), 127.8 (CH), 130.2 (CH), 131.9 (C), 142.2 (C), 143.5 (C), 143.7 (C), 158.2 (C) ppm. IR (HATR): 2952, 2936, 2874, 2844, 1695, 1468, 1364, 1338, 1290, 1228, 1152, 1078, 1025, 1016, 775, 726 cm⁻¹. EI-MS m/z (rel. intensity %): 421 (M⁺, <1), 287 (100), 258 (11), 154 (20), 90 (21). ES-MS: 421 [M+H]⁺. [α]_{D}²⁰=−157.3 (c 0.56, CHCl₃). Mp: 184-186° C. HRMS (EI) calculated for C₂₈H₂₄O₂N₂ 420.1838; found 420.1830.

(R)-Indan-1-yl-(3H-isobenzofuran-1-ylidene)-amine (I_{g}). A suspension of (R)-(−)-indan-1-yl-amine (100 mg, 0.75 mmol) and imidate II-A (178.0 mg, 1.05 mmol) in dry CH₂Cl₂ (5 mL) was cooled in an ice bath. Et₃N (0.31 mL, 2.25 mmol) was added and the resulting suspension was stirred for 24 h at room temperature. Evaporation in vacuo and purification by flash chromatography over silica gel (toluene/Et₂O, 6/4, +1% Et₃N) resulted in I_{g} as a white solid, 138 mg (74%). ¹H-NMR (300 MHz, CDCl₃) δ 2.09-2.21 (m, J=7.5, 8.7, 12.5 Hz, 1H), 2.59-2.64 (m, J=3.2, 7.5, 12.5 Hz, 1H), 2.93-3.04 (m, J=15.7 Hz, 1H), 3.11-3.20 (ddd, J=3.2, 8.8, 15.7 Hz, 1H), 5.42 (s, 2H), 5.57 (dd, J=7.5, 7.5 Hz, 1H), 7.20-7.45 (m, 5H), 7.48 (d, J=7.5 Hz, 1H), 7.55 (t, J=7.5 Hz, 1H), 7.96 (d, J=7.5 Hz, 1H) ppm. ¹³C-NMR (75.4 MHz, CDCl₃): 30.8 (CH₂), 34.6 (CH₂), 61.2 (CH), 72.0 (CH₂), 121.2 (CH), 123.6 (CH), 124.2 (CH), 124.4 (CH), 126.2 (CH), 126.8 (CH), 128.3 (CH), 130.5 (C), 131.1 (CH), 143.1 (C), 143.4 (C), 145.8 (C), 160.1 (C) ppm. IR (HATR): 3018, 2957, 2931, 2859, 1689, 1470, 1456, 1361, 1331, 1289, 1073, 1024, 1015, 1002, 781, 776, 766, 740, 726, 700, 670 cm⁻¹. EI-MS m/z (rel. intensity %): 249 (M⁺, 20), 234 (11), 220 (13), 134 (100), 118 (64), 90 (80), 76 (16), 63 (27), 51 (21). [α]_{D}²⁰=+123.5 (c 0.78, CHCl₃). Mp: 79-80° C. HRMS (EI) calculated for C₁₇H₁₅ON 249.1154; found 249.1154.

N,N′-bis-(7-chloro-3H-isobenzofuran-1-ylidene)-cyclohexane-(1R,2R)-diamine (Iₕ). A suspension of (1R,2R)-(−)-diaminocyclohexane (71.5 mg, 0.63 mmol) and imidate II-B (325 mg, 1.59 mmol) in dry CH₂Cl₂ (3 mL) was cooled in an ice bath. Et₃N (1.1 mL, 7.97 mmol) was added and the resulting suspension was refluxed for 24 h. The reaction mixture was passed through a short pad of silica gel and eluted with EtOAc. Evaporation in vacuo and purification by flash chromatography over silica gel (toluene/Et₂O, 6/4, +1% Et₃N) resulted in (Iₕ) as a white solid, 114 mg (44%). ¹H-NMR (300 MHz, CDCl₃) δ 1.47-1.71 (m, 4H), 1.79-1.85 (m, 2H), 1.98-2.02 (m, 2H), 4.04 (dd, J=3.7, 4.9 Hz, 2H), 5.21 (s, 4H), 7.15-7.17 (m, 2H), 7.29-7.30 (m, 4H) ppm. ¹³C-NMR (75.4 MHz, CDCl₃): 24.6 (CH₂), 31.9 (CH₂), 61.4 (CH), 70.4 (CH₂), 119.6 (CH), 127.3 (C), 129.9 (CH), 130.9 (C), 131.1 (C), 145.9 (C), 155.7 (C) ppm. IR (HATR): 2928, 2871, 2855, 1681, 1606, 1585, 1462, 1361, 1307, 1244, 1221, 1176, 1145, 1092, 1021, 913, 772, 729, 661 cm⁻¹. EI-MS m/z (rel. intensity %): 414 (M+, 15), 379 (13), 247 (15), 206 (13), 168 (51), 152 (86), 126 (28), 124 (64), 89 (100). [α]_{D}²⁰=−26.1 (c 0.95, CHCl3). Mp: 62° C.

N,N′-bis-(5-chloro-3H-isobenzofuran-1-ylidene)-cyclohexane-(1R,2R)-diamine (I₁). A suspension of (1R,2R)-(−)-diaminocyclohexane (70.0 mg, 0.61 mmol) and imidate II-C (325 mg, 1.59 mmol) in dry CH₂Cl₂ (3 mL) was cooled in an ice bath. Et₃N (1.1 mL, 7.97 mmol) was added and the resulting suspension was refluxed for 24 h. The reaction mixture was passed through a short pad of silica gel and eluted with EtOAc. Evaporation in vacuo and purification by flash chromatography over silica gel (toluene/Et₂O, 6/4, +1% Et₃N) resulted in (II) as a white solid, 190.8 mg (75%). ¹H-NMR (300 MHz, CDCl₃) δ 1.40-1.47 (m, 2H), 1.50-1.61 (m, 2H), 1.77-1.80 (m, 2H), 1.87-1.91 (m, 2H), 3.91 (dd, J=3.7, 5.1 Hz, 2H), 5.12 (d, J=14.5 Hz, 2H), 5.20 (d, J=14.5 Hz, 2H), 7.22-7.24 (m, 2H), 7.26-7.27 (m, 2H), 7.28-7.29 (m, 2H) ppm. ¹³C-NMR (75.4 MHz, CDCl₃): 24.8 (CH2), 32.3 (CH2), 61.1 (CH), 71.0 (CH2), 121.5 (CH), 124.4 (CH), 128.6 (CH), 129.4 (C), 136.8 (C), 144.6 (C), 157.6 (C) ppm. IR (HATR): 2936, 2876, 1690, 1611, 1469, 1453, 1423, 1350, 1334, 1304, 1281, 1260, 1220, 1190, 1158, 1088, 1058, 1026, 1007, 978, 886, 867, 838, 781, 740, 713, 700, 671, 659 cm⁻¹. EI-MS m/z (rel. intensity %): 414 (M⁺, 16), 247 (17), 234 (7), 206 (14), 194 (24), 168 (36), 152 (74), 124 (62), 89 (100). [α]_{D}²⁰=+60.1 (c 1.08, CHCl₃). Mp: 212° C.

TABLE 3
synthesis of chiral imidates

| | | Imidate | Product | Yield |
|---|---|---|---|---|
| Entry | Amine (R—NH₂) | (II) | (I) | (%) |
| 1 | (1R,2R)-(−)-1,2-diaminocyclohexane | II-A | Iₐ | 85 |
| 2^{b} | (R)-(+)-1,1′-binaphthyl-2,2′-diamine | II-A | I_{b} | 71 |
| 3 | (1S,2S,4S,5S)-2,5-diamino-norbornane | II-A | I_{c} | 56 |
| 4 | (4S,5S)-4,5-di(aminomethyl)-2,2- | II-A | I_{d} | 92 |
| | dimethyl-1,3-dioxolane | | | |
| 5 | (1R,8R)-1,2,3,6,7,8-hexahydro-as- | II-A | Iₑ | 93 |
| | indacene-1,8-diamine | | | |
| 6^{c} | (1R,2R)-(−)-trans-1-amino-2-indanol | II-A | I_{f} | 91 |
| 7^{c} | R-(−)-aminoindane | II-A | I_{g} | 74 |
| 8 | (1R,2R)-(−)-diaminocyclohexane | II-B | Iₕ | 44 |
| 9 | (1R,2R)-(−)-diaminocyclohexane | II-C | Iᵢ | 75 |
| 10^{c} | (Sₚ)-1-[(1R)-(1-aminoethyl)]-2- | II-A | Iⱼ | 97 |
| | (diphenylphosphino)ferrocene | | | |
| 11^{c} | (Sₚ)-1-[(1R)-(1-aminoethyl)]-2- | II-C | Iₖ | 79 |
| | (diphenylphosphino)ferrocene | | | |
| 12^{c} | (Sₚ)-1-[(1R)-(1-aminoethyl)]-2- | II-B | Iₗ | 61 |
| | (diphenylphosphino)ferrocene | | | |
| 13^{c} | (Sₚ)-1-[(1R)-(1-aminoethyl)]-2- | II-D | Iₘ | 51 |
| | (diphenylphosphino)ferrocene | | | |
| 14^{c} | (Sₚ)-1-[(1R)-(1-aminoethyl)]-2- | II-E | Iₙ | 95 |
| | (diphenylphosphino)ferrocene | | | |
| 15^{c} | (Sₚ)-1-[(1R)-(1-aminoethyl)]-2- | II-G | Iₒ | 89 |
| | (diphenylphosphino)ferrocene | | | |
| 16^{c} | (Sₚ)-1-[(1R)-(1-aminoethyl)]-2- | II-H | Iₚ | 42 |
| | (diphenylphosphino)ferrocene | | | |

^{a}Reagents and conditions: 2 (1 equiv), 1 (2.6 equiv), Et₃N (13 equiv), CH₂Cl₂,
^{b}reflux in EtOH,
^{c}1.3 equiv of 1.

The ligands were perfectly stable for a long period at room temperature. In particular, ligand IIb showed no sign of decomposition after 1 month in CDCl₃ at room temperature.

I.2 Imidate Ligands from Aminoalcohols

In a preferred embodiment, the cyclic imidates of formula (I) are obtained from an aminoalcohol, preferably a chiral non-racemic aminoalcohol. In a more preferred embodiment, the cyclic imidates of formula (I) are obtainable or obtained from (1R,2R)-trans-1-amino-2-indanol. Imidate alcohol ligands are obtainable as described below. Note that when X—NH₂ is an aminoalcohol, a ligand corresponding with formula I is formed, which may rearrange into a ligand of formula X, depending on the structure (Scheme 1).

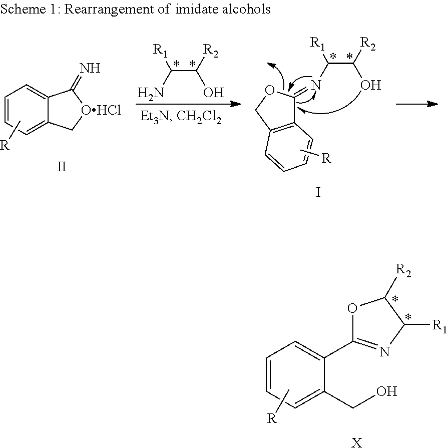

(1R,2R)-Trans-1-(3H-isobenzofuran-1-ylideneamino)-indan-2-ol (I_{f}). A suspension of (1R,2R)-(−)-trans-1-amino-2-indanol (100.0 mg, 0.67 mmol) and imidate ester IIA (125.0 mg, 0.74 mmol) in CH₂Cl₂ was cooled in an ice bath. Et₃N (0.28 mL, 2.0 mmol) was added and the reaction mixture was stirred for 48 h at room temperature. Evaporation in vacuo and recrystallization from CH₂Cl₂ resulted in I_{f} as a white solid, 161 mg (91%). ¹H-NMR (300 MHz, DMSO-d₆) δ 2.74 (dd, J=7.0, 15.6 Hz, 1H), 3.18 (dd, J=7.0, 15.6 Hz, 1H), 4.33 (m, J=5.6, 7.0 Hz, 1H), 5.12 (d, J=5.6 Hz, 1H), 5.16 (d, J=5.2 Hz, 1H), 5.44 (d, J=14.9 Hz, 1H), 5.50 (d, J=14.9 Hz, 1H), 7.05-7.20 (m, 4H), 7.44-7.49 (m, 1H), 7.56-7.63 (m, 2H), 7.70 (d, J=7.6 Hz, 1H) ppm. ¹³C-NMR+HSQC (75.4 MHz, DMSO-d₆): 39.3 (CH₂), 68.3 (CH), 72.1 (CH₂), 79.5 (CH), 122.2 (CH), 122.8 (CH), 124.3 (CH), 124.5 (CH), 126.4 (CH), 127.1 (CH), 128.4 (CH), 129.7 (C), 131.5 (CH), 140.2 (C), 143.2 (C), 143.8 (C), 160.1 (C) ppm. IR (HATR): 3189, 1680, 1467, 1419, 1369, 1298, 1225, 1200, 1084, 1028, 998, 777, 747, 730, 703, 675 cm⁻¹. EI-MS m/z (rel. intensity %): 265 (M⁺, 20), 247 (4), 237 (17), 218 (5), 146 (15), 134 (23), 118 (100), 104 (50), 90 (97), 63 (19), 49 (43). ES-MS: 266 [M+H]⁺. [α]_{D}²⁰=−304.8 (c 0.81, DMSO-d₆). Mp: 236° C. HRMS (EI) calculated for C₁₇H₁₅O₂N 265.1103; found 265.1107.

I.3 Imidate Ligands from Aminophosphines

In a preferred embodiment, the cyclic imidates of formula (I) are obtained from an aminophosphine, preferably a chiral aminophosphine. In a more preferred embodiment, the cyclic imidates of formula (I) are obtainable or obtained from (Rp)-1-[(1S)-(1-aminoethyl)]-2-(diphenylphosphino)ferrocene.

(Sₚ)-1-[(1R)-(1-(3H-isobenzofuran-1-ylideneamino)-ethyl)]-2-(diphenylphosphino)-ferrocene (Iⱼ). A suspension of (Sₚ)-1-[(1R)-(1-aminoethyl)]-2-(diphenylphosphino) ferrocene (70.0 mg, 0.17 mmol) and imidate II-A (44 mg, 0.26 mmol) in dry CH₂Cl₂ (2 mL) was cooled in an ice bath. Et₃N (80.0 μL, 0.57 mmol) was added and the resulting suspension was refluxed for 48 h. Evaporation in vacuo and purification by flash chromatography over silica gel (hexane/EtOAc, 7/3) resulted in (Iⱼ) as a brownish oil, 87.0 mg (97%). ¹H-NMR (300 MHz, CDCl₃) δ 1.62 (d, J=6.6 Hz, 3H), 3.62-3.65 (m, 1H), 4.08 (s, 5H), 4.26-4.28 (m, 1H), 4.65 (m, 1H), 4.83 (d, J=14.2 Hz, 1H), 5.10 (d, J=14.2 Hz, 1H), 5.36-5.43 (m, 1H), 6.59-6.64 (m, 1H), 6.72-6.77 (m, 2H), 6.97-7.02 (m, 2H), 7.06-7.16 (m, 2H), 7.27-7.33 (m, 5H), 7.45-7.51 (m, 2H) ppm. ¹³C-NMR (75.4 MHz, CDCl₃): 20.67 (CH₃), 49.58 (CH₂, J_{CP}=8.8 Hz), 68.7 (CH, J_{CP}=4.0 Hz), 68.8 (CH), 68.5 (5xCH), 71.3 (CH, J_{CP}=4.5 Hz), 71.8 (C), 75.3 (C, J_{CP}=6.6 Hz), 98.3 (C, J_{CP}==23.9 Hz), 120.5 (CH), 123.6 (CH), 126.8 (CH), 127.0 (CH, J_{CP}=6.3 Hz), 127.4 (CH), 127.9 (CH, J_{CP}=7.7 Hz), 128.8 (CH), 129.8 (C), 130.4 (CH), 131.8 (CH), 132.1 (CH), 135.2 (CH, J_{CP}=20.9 Hz), 137.6 (C, J_{CP}=8.6 Hz), 139.2 (C, J_{CP}=9.4 Hz), 142.8 (C), 145.4 (C), 158.0 (C) ppm. ³¹P-NMR (121.4 MHz, CDCl₃): −22.5 ppm. IR (HATR): 3050, 2972, 2931, 2873, 1681, 1469, 1451, 1433, 1363, 1290, 1243, 1167, 1106, 1081, 1044, 1017, 1000, 819, 747, 728, 697 cm⁻¹. EI-MS m/z (rel. intensity %): 529 (M+, 8), 396 (19), 275 (8), 212 (9), 183 (17), 165 (15), 133 (11), 121 (100), 77 (17), 56 (30). ES-MS: 530 [M+H]⁺. [α]_{D}²⁰=−338.8 (c 0.64, CHCl₃).

(Sₚ)-1-[(1R)-(1-(5-chloro-3H-isobenzofuran-1-ylideneamino)-ethyl)]-2-(diphenylphosphino)-ferrocene (Iₖ). A suspension of (Sₚ)-1-[(R1R)-(1-aminoethyl)]-2-(diphenyl-phosphino)ferrocene (100.0 mg, 0.24 mmol) and imidate II-C (64.2 mg, 0.31 mmol) in dry CH₂Cl₂ (2.5 mL) was cooled in an ice bath. Et₃N (102.0 μL, 0.73 mmol) was added and the resulting suspension was refluxed for 48 h. Evaporation in vacuo and purification by flash chromatography over silica gel (hexane/EtOAc, 8/2) resulted in (Iₖ) as a brownish oil, 107.0 mg (79%). ¹H-NMR (300 MHz, CDCl₃) δ 1.63 (d, J=6.6 Hz, 3H), 3.66 (m, 1H), 4.11 (s, 5H), 4.30 (s, 1H), 4.67 (m, 1H), 4.83 (d, J=14.4 Hz, 1H) 5.09 (d, J=14.4 Hz, 1H), 5.37-5.44 (m, 1H), 6.70-6.75 (m, 1H), 6.80-6.84 (m, 2H), 6.99-7.04 (m, 2H), 7.09-7.28 (m, 3H), 7.34-7.35 (m, 3H), 7.47-7.53 (m, 2H) ppm. ¹³C-NMR (75.4 MHz, CDCl₃): 20.5 (CH₃), 49.8 (CH₂, J_{CP}=8.8 Hz), 68.7 (CH, J_{CP}=4.0 Hz), 68.9 (CH), 69.5 (5×CH), 71.1 (C), 71.4 (CH, J_{CP}=4.4 Hz), 75.3 (C, J_{CP}=6.6 Hz), 98.2 (C, J_{CP}=23.7 Hz), 120.9 (CH), 124.7 (CH), 126.9 (CH), 127.1 (CH, J_{CP}=6.2 Hz), 127.9 (CH, J_{CP}=7.7 Hz), 128.03 (CH), 128.7 (C), 128.8 (CH), 132.0 (CH, J_{CP}=18.6 Hz), 135.2 (CH, J_{CP}=20.9 Hz), 136.5 (C), 137.5 (C, J_{CP}=8.7 Hz), 139.3 (C, J_{CP}=9.8 Hz), 144.4 (C), 156.5 (C). ³¹P-NMR (121.4 MHz, CDCl₃): −22.6 ppm. IR (HATR): 3067, 2969, 2931, 2871, 2358, 2341, 1689, 1613, 1473, 1456, 1432, 1354, 1304, 1265, 1242, 1222, 1192, 1167, 1106, 1080, 1042, 1018, 879, 822, 742, 697, 668 cm⁻¹. ES-MS: 564 [M+H]⁺. [α]_{D}²⁰=−338.1 (c 0.64, CHCl₃).

(Sₚ)-1-[(1R)-(1-(7-chloro-3H-isobenzofuran-1-ylideneamino)-ethyl)]-2-(diphenylphosphino)-ferrocene (Iₗ). A suspension of (Sₚ)-1-[(1R)-(1-aminoethyl)]-2-(diphenyl-phosphino)-ferrocene (100.0 mg, 0.24 mmol) and imidate II-B (64.2 mg, 0.31 mmol) in dry CH₂Cl₂ (2.5 mL) was cooled in an ice bath. Et₃N (102.0 μL, 0.73 mmol) was added and the resulting suspension was refluxed for 48 h. Evaporation in vacuo and purification by flash chromatography over silica gel (hexane/EtOAc, 85/15) resulted in I_{I} as a brownish oil, 80.9 mg (61%). ¹H-NMR (300 MHz, CDCl₃) δ 1.64 (d, J=6.6 Hz, 3H), 3.62 (m, 1H), 4.08 (s, 5H), 4.27 (m, 1H), 4.65 (m, 1H), 4.72 (d, J=14.3 Hz, 1H) 5.01 (d, J=14.3 Hz, 1H), 5.33-5.41 (m, 1H), 6.54-6.59 (m, 1H), 6.75-6.80 (m, 2H), 6.93-7.02 (m, 3H), 7.14-7.21 (m, 2H), 7.30-7.35 (m, 3H), 7.45-7.52 (m, 2H) ppm. ¹³C-NMR (75.4 MHz, CDCl₃): δ 21.1 (CH₃), 50.0 (d, J_{CP}=8.8 Hz, CH), 68.8 (d, J_{CP}=3.9 Hz, CH), 68.9 (CH), 69.5 (5×CH), 70.4 (CH₂), 71.0 (d, J_{CP}=4.3 Hz, CH), 75.1 (d, J_{CP}=6.1 Hz, C), 99.0 (d, J_{CP}=24.2 Hz, C), 118.9 (CH), 126.6 (CH), 126.7 (C), 127.0 (d, J_{CP}=6.1 Hz, CH), 127.9 (d, J_{CP}=7.7 Hz, CH), 128.8 (CH), 129.5 (CH), 130.8 (CH), 131.2 (C), 131.9 (d, J_{CP}=18.3 Hz, CH), 135.3 (d, J_{CP}=21.0 Hz, CH), 137.7 (d, J_{CP}=8.8 Hz, C), 139.2 (d, J_{CP}=9.9 Hz, C), 145.6 (C), 154.5 (C) ppm. ³¹P-NMR (121.4 MHz, CDCl₃): −22.0 ppm. IR (HATR): 3054, 2972, 2931, 1678, 1606, 1585, 1478, 1462, 1433, 1361, 1306, 1265, 1244, 1220, 1167, 1106, 1078, 1040, 1026, 1000, 915, 818, 774, 738, 698, 668 cm⁻¹. EI-MS m/z (rel. intensity %): 563 (M⁺, 7), 396 (100), 331 (21), 288 (21), 252 (17), 226 (6), 183 (20), 167 (32), 138 (60), 102 (31), 75 (24), 56 (52). ES-MS: 564 [M+H]⁺. [α]_{D}²⁰=−367.6 (c 0.70, CHCl₃). HRMS (EI): calcd for C₃₂H₂₇NOP³⁵CIFe: 563.0868; found 563.0857.

(Sₚ)-1-[(1R)-(1-(7-bromo-3H-isobenzofuran-1-ylideneamino)-ethyl)]-2-(diphenylphosphino)-ferrocene (Iₘ). A suspension of (Sₚ)-1-[(1R)-(1-aminoethyl)]-2-(diphenyl-phosphino)-ferrocene (98.0 mg, 0.24 mmol) and imidate II-D (77.0 mg, 0.31 mmol) in dry CH₂Cl₂ (2.5 mL) was cooled in an ice bath. Et₃N (102.0 μL, 0.73 mmol) was added and the resulting suspension was refluxed for 48 h. Evaporation in vacuo and purification by flash chromatography over silica gel (hexane/EtOAc, 85/15) resulted in Iₘ as a brownish oil, 74.5 mg (51%). ¹H-NMR (300 MHz, CDCl₃) δ 1.64 (d, J=6.6 Hz, 3H), 3.61 (m, 1H), 4.09 (s, 5H), 4.27 (m, 1H), 4.65-4.70 (m, 2H), 4.99 (d, J=14.3 Hz, 1H), 5.31-5.39 (m, 1H), 6.53-6.58 (m, 1H), 6.75-6.81 (m, 2H), 6.97-7.02 (m, 3H), 7.08-7.13 (m, 1H), 7.31-7.51 (m, 6H) ppm. ¹³C-NMR (75.4 MHz, CDCl₃): δ 21.3 (CH₃), 49.8 (d, J_{CP}=8.7 Hz, CH), 68.8 (d, J_{CP}=4.0 Hz, CH), 68.9 (CH), 69.5 (5×CH), 70.1 (CH₂), 71.0 (d, J_{CP}=4.4 Hz, CH), 75.0 (d, J_{CP}=6.1 Hz, C), 99.1 (d, J_{CP}=23.9 Hz, C), 119.2 (C), 119.6 (CH), 126.5 (CH), 127.1 (d, J_{CP}=6.2 Hz, CH), 127.9 (d, J_{CP}=7.7 Hz, CH), 128.2 (C), 128.9 (CH), 130.9 (CH), 131.9 (d, J_{CP}=18.3 Hz, CH), 132.9 (CH), 135.3 (d, J_{CP}=21.0 Hz, CH), 137.8 (d, J_{CP}=8.9 Hz, C), 139.2 (d, J_{CP}=10.0 Hz, C), 145.7 (C), 154.4 (C) ppm. ³¹P-NMR (121.4 MHz, CDCl₃): −22.0 ppm. IR (HATR): 3052, 2971, 2930, 1680, 1580, 1478, 1458, 1433, 1361, 1321, 1303, 1266, 1244, 1217, 1106, 1079, 1039, 1000, 892, 819, 774, 741, 696, 668 cm⁻¹. EI-MS m/z (rel. intensity %): 607 (M⁺, 5), 396 (100), 331 (22), 319 (10), 288 (22), 252 (18), 211 (20), 182 (34), 165 (27), 121 (57), 102 (27), 56 (55). ES-MS: 607.9 [M+H]⁺. [α]_{D}²⁰=−322.2 (c 0.99, CHCl₃). HRMS (EI): calcd for C₃₂H₂₇NOP⁷⁹BrFe: 607.0363; found 607.0382.

(Sₚ)-1-[(1R)-(1-(5,6-dimethoxy-3H-isobenzofuran-1-ylideneamino)-ethyl)]-2-(diphenylphosphino)-ferrocene (Iₙ). A suspension of (Sₚ)-1-[(1R)-(1-aminoethyl)]-2-(diphenyl-phosphino) ferrocene (300 mg, 0.726 mmol) and imidate II-E (216.8 mg, 0.944 mmol) in dry CH₂Cl₂ (5 mL) was cooled in an ice bath. Et₃N (303.5 μL, 2.18 mmol) was added and the resulting suspension was refluxed for 48 h. Evaporation in vacuo and purification by flash chromatography over silica gel (hexane/EtOAc, 70/30) resulted in Iₙ as a brownish oil, 406.7 mg (95%). ¹H-NMR (300 MHz, CDCl₃) δ 1.61 (d, J=6.4 Hz, 3H), 3.65 (s, 1H), 3.79 (s, 3H), 3.86 (s, 3H), 4.06 (s, 5H), 4.28 (s, 1H), 4.65 (s, 1H), 4.80 (d, J=13.8 Hz, 1H), 5.05 (d, J=13.8 Hz, 1H), 5.36-5.43 (m, 1H), 6.56 (s, 1H), 6.70-6.82 (m, 4H), 6.97-7.02 (t, J=7.15 Hz, 2H), 7.30-7.32 (m, 3H), 7.44-7.50 (t, J=7.15 Hz, 2H) ppm. ³¹P-NMR (121.4 MHz, CDCl₃): −22.7 ppm. IR (HATR): 3067, 2923, 1734, 1682, 1620, 1606, 1586, 1500, 1472, 1433, 1353, 1286, 1243, 1224, 1192, 1165, 1135, 1106, 1081, 1041, 1018, 939, 911, 859, 820, 774, 740, 696, 654 cm⁻¹. [α]_{D}²⁰=−353.9 (c 0.99, CHCl₃).

(Sₚ)-1-[(1R)-(1-(6-methyl-3H-isobenzofuran-1-ylideneamino)-ethyl)]-2-(diphenylphosphino)-ferrocene (Iₒ). A suspension of (Sₚ)-1-[(1R)-(1-aminoethyl)]-2-(diphenyl-phosphino)-ferrocene (300 mg, 0.726 mmol) and imidate II-G (173.3 mg, 0.944 mmol) in dry CH₂Cl₂ (5 mL) was cooled in an ice bath. Et₃N (303.5 μL, 2.18 mmol) was added and the resulting suspension was refluxed for 48 h. Evaporation in vacuo and purification by flash chromatography over silica gel (hexane/EtOAc, 80/20) resulted in Iₒ as a brownish oil, 353 mg (89%). ¹H-NMR (300 MHz, CDCl₃) δ 1.61 (s, 3H), 2.26 (s, 3H), 3.64 (s, 1H), 4.07 (s, 5H), 4.27 (s, 1H), 4.65 (s, 1H), 4.83 (d, J=13.4 Hz, 1H), 5.09 (d, J=13.4 Hz, 1H), 5.37-5.40 (m, 1H), 6.66-6.68 (t, J=6.9 Hz, 1H), 6.76-6.78 (t, J=7.3 Hz, 2H), 6.96-7.00 (m, 3H), 7.05 (s, 1H), 7.10 (d, J=6.9 Hz, 1H), 7.30-7.33 (m, 3H), 7.46-7.48 (t, J=7.3 Hz, 2H) ppm. ¹³C-NMR (75.4 MHz, CDCl₃): δ . . . ³¹P-NMR (121.4 MHz, CDCl₃): −22.7 ppm. IR (HATR): 3050, 2926, 1734, 1678, 1500, 1472, 1433, 1354, 1278, 1242, 1194, 1162, 1106, 1091, 1069, 1036, 1016, 939, 867, 816, 773, 741, 696, 654 cm⁻¹. ES-MS: 544 [M+H]⁺. [α]_{D}²⁰−388.9 (c 1.02, CHCl₃).

(Sₚ)-1-[(1R)-(1-(5,6-methylenedioxy-3H-isobenzofuran-1-ylideneamino)-ethyl)]-2-(diphenylphosphino)-ferrocene (Iₙ). A suspension of (Sₚ)-1-[(1R)-(1-aminoethyl)]-2-(diphenylphosphino) ferrocene (300 mg, 0.726 mmol) and imidate II-H (167.2 mg, 0.944 mmol) in dry CH₂Cl₂ (5 mL) was cooled in an ice bath. Et₃N (303.5 μL, 2.18 mmol) was added and the resulting suspension was refluxed for 48 h. Evaporation in vacuo and purification by flash chromatography over silica gel (hexane/EtOAc, 70/30) resulted in I p as a brownish oil, 231.7 mg (42%). ¹H-NMR (300 MHz, CDCl₃) δ 1.59 (d, J=6.7 Hz, 3H), 2.03 (s, 2H), 3.62-3.65 (m, 1H), 4.07 (s, 5H), 4.26-4.27 (m, 1H), 4.63 (s, 1H), 4.73 (d, J=14.1 Hz, 1H), 4.99 (d, J=14.1 Hz, 1H), 5.29-5.37 (m, 1H), 6.48 (s, 1H), 6.64 (s, 1H), 6.75-6.88 (m, 3H), 6.98-7.03 (t, J=7.3 Hz, 2H), 7.30-7.32 (m, 3H), 7.44-7.50 (t, J=7.3 Hz, 2H) ppm. ³¹P-NMR (121.4 MHz, CDCl₃): −22.6 ppm. IR (HATR): 3070, 2921, 1760, 1736, 1678, 1501, 1472, 1433, 1354, 1278, 1242, 1193, 1162, 1106, 1091, 1069, 1035, 1016, 939, 868, 815, 774, 741, 696 cm⁻¹. ES-MS: 574 [M+H]⁺. [α]_{D}²⁰=−363.7 (c 1.01, CHCl₃).

II. Catalysts comprising Imidates

In another aspect of the invention a catalyst is provided, wherein the catalyst is formed by complexing a catalyst precursor with an imidate of the invention.

II.1. Metal Catalysts

In another aspect of the invention a catalyst is provided, wherein the catalyst is formed by complexing a catalyst precursor comprising a metal with an imidate of the invention. A metal is preferably selected from a group comprising, but not limited to copper, palladium, nickel, platinum, zinc, rhodium, ruthenium, manganese, iron, aluminium, magnesium.

In a preferred embodiment, the metal is copper. In a more preferred embodiment, the catalyst is Cu(Iₐ)₂PF₆.

Molecular modelling of bisimidate Iₐ revealed that the imidate groups are axially orientated (FIG. 4). This was confirmed by ¹H-NMR: the alpha-protons showed two small vicinal coupling constants (dd, J=3.9, 4.7 Hz) suggesting a trans-diequatorial relationship.

Cu(Iₐ)₂PF₆ was obtained as follows: c-Hexane-bisimidate Iₐ (31.0 mg, 89.5 μmol) and Cu(MeCN)₄PF₆ (29.4 mg, 78.9 μmol) were dissolved in acetonitrile (2 mL). The resulting yellow suspension was filtrated and evaporated in vacuo. The resulting yellow solids were recrystallized from benzene. This resulted in pure Cu(Iₐ)₂PF₆ as a yellow solid, 40.3 mg (quantitative yield). ¹H-NMR (300 MHz, CD₃CN) δ 1.20 (m, 8H), 1.68 (m, 4H), 2.31 (d, J=10.4 Hz, 4H), 3.20 (br s, 4H), 4.83 (d, J=15.4 Hz, 4H), 5.23 (d, J=15.4 Hz, 4H), 7.42 (m, 8H), 7.58 (t, J=7.5 Hz, 4H), 8.27 (d, J=7.5 Hz, 4H) ppm. ¹³C-NMR (75.4 MHz, CD₃CN): 26.0 (CH₂), 31.7 (CH₂), 64.0 (CH), 75.2 (CH₂), 122.8 (CH), 125.4 (CH), 129.3 (CH), 130.0 (C), 133.5 (CH), 144.8 (C), 167.0 (C) ppm. IR (HATR): 2937, 2861, 1644, 1470, 1452, 1364, 1298, 1102, 1095, 1040, 1020, 998, 953, 832, 776, 726, 673 cm⁻¹. ES-MS: 755 [Cu(Iₐ)₂]⁺, 450 [Cu(Iₐ) CH₃CN]⁺, 409 [Cu(Iₐ)]⁺, 347 [Iₐ+H]⁺. [α]_{D}²⁰=−387.3 (c 0.79, CH₃CN). Mp: decomposition at 245° C.

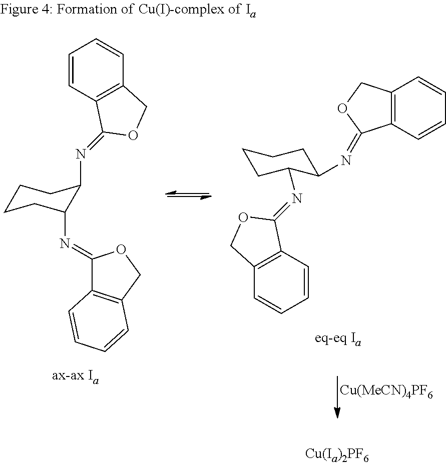

Suitable crystals for X-ray diffraction were grown from a solution of the complex in MTBE/CH₃CN. An X-ray structure was obtained, shown in FIG. 5. This revealed that in the complex the opposite chair conformation is adopted, with the imidate groups in equatorial position and hence suitable for complexation with Cu(I). The Cu(I) complex shows a tetrahedral arrangement with two ligands around the metal. The Cu—N bond lengths are 2.07 Å and 2.05 Å for both ligands. The angles between N(2)-Cu(1)-N(9) and N(28)-Cu(1)-N(35) are respectively 84.0° and 84.4°. The imidate groups clearly possess the (Z)-geometry dissecting the space around the metal effectively in a C₂-fashion.

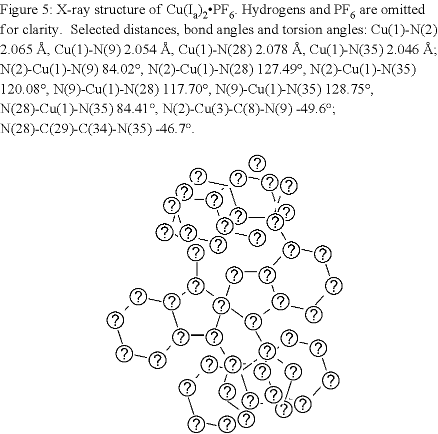

In another preferred embodiment, the metal is iridium. In a more preferred embodiment, the catalyst is [Ir(Ij-Ip)COD]BarF.

### Iridium Complex of Ligand Iⱼ

In a Schlenk tube under an argon atmosphere, a mixture of ligand (Sₚ)-1-[(1R)-(1-(3H-Isobenzofuran-1-ylideneamino)-ethyl)]-2-(diphenylphosphino)-ferrocene (Iⱼ). (20 mg, 0.0355 mmol) and [Ir(COD)Cl]₂ (12 mg, 0.0177 mmol) in dry CH₂Cl₂ (1 mL) was refluxed and stirred during 2 h. The solvent was evaporated. Purification by flash chromatography over silica gel (pentane/CH₂Cl₂, 50/50) resulted in an orange foaming solid, 58 mg (91%). ³¹P-NMR (121.4 MHz, CDCl₃): 7.2 ppm. ES-MS: 830.1 [M-BARF]⁺

Iridium complex of ligand Iₖ

In a Schlenk tube under an argon atmosphere, a mixture of ligand (Sₚ)-1-[(1R)-(1-(5-chloro-3H-isobenzofuran-1-ylideneamino)-ethyl)]-2-(diphenylphosphino)-ferrocene (Iₖ) (20 mg, 0.0355 mmol) and [Ir(COD)Cl]₂ (12 mg, 0.0177 mmol) in dry CH₂Cl₂ (1 mL) was refluxed and stirred during 2 h. After cooling down to room temperature, NaBARF (47 mg, 0.0532 mmol) was added to the solution and stirred for 5 min. Then, H₂O (1 mL) was added, and the mixture was stirred vigorously for 15 min. The organic layer was seperated, and the aqueous phase is was extracted with CH₂Cl₂ (2×1 mL). The combined organic phases were dried over MgSO₄, filtered and concentrated under reduced pressure. Purification by flash chromatography over silica gel (pentane/CH₂Cl₂, 50/50) resulted in an orange foaming solid, 59 mg (97%). ³¹P-NMR (121.4 MHz, CDCl₃): 7.4 ppm. ES-MS: 864.0 [M-BARF]⁺

### Iridium Complex of Ligand Iₙ:

In a Schlenk tube under an argon atmosphere, a mixture of ligand (Sₚ)-1-[(1R)-(1-(5,6-dimethoxy-3H-isobenzofuran-1-ylideneamino)-ethyl)]-2-(diphenyl phosphino)-ferrocene (Iₙ) (100 mg, 0.170 mmol) and [Ir(COD)Cl]₂ (57 mg, 0.0848 mmol) in dry CH₂Cl₂ (5 mL) was refluxed and stirred during 2 h. After cooling down to room temperature, NaBARF (225.5 mg, 0.254 mmol) was added to the solution and stirred for 5 min. Then, H₂O (5 mL) is added, and the mixture was stirred vigorously for 20 min. The organic layer was seperated, and the aqueous phase was extracted with CH₂Cl₂ (2×5 mL). The combined organic phases were dried over MgSO₄, filtered and concentrated under reduced pressure. Purification by flash chromatography over silica gel (pentane/CH₂Cl₂, 50/50) resulted in an orange foaming solid, 261.1 mg (88%). ³¹P-NMR (121.4 MHz, CDCl₃): +6.8 ppm. IR (HATR): 2890, 1610, 1498, 1461, 1353, 1296, 1273, 1228, 1118, 1081, 1059, 1032, 1001, 886, 839, 744, 712, 682, 669 cm⁻¹. ES-MS: 890.1 [M-BARF]⁺

Iridium complex of ligand Iₒ

In a Schlenk tube under an argon atmosphere, a mixture of ligand (Sₚ)-1-[(1R)-(1-(6-methyl-3H-isobenzofuran-1-ylideneamino)-ethyl)]-2-(diphenylphosphino)-ferrocene (Iₛ) (94.7 mg, 0.174 mmol) and [Ir(COD)Cl]₂ (62.3 mg, 0.0928 mmol) in dry CH₂Cl₂ (5 mL) was refluxed and stirred during 2 h. After cooling down to room temperature, NaBARF (248.5 mg, 0.280 mmol) was added to the solution and stirred for 5 min. Then, H₂O (5 mL) was added, and the mixture was stirred vigorously for 20 min. The organic layer was seperated, and the aqueous phase is was extracted with CH₂Cl₂ (2×5 mL). The combined organic phases were dried over MgSO₄, filtered and concentrated under reduced pressure. Purification by flash chromatography over silica gel (pentane/CH₂Cl₂, 50/50) resulted in an orange foaming solid, 269 mg (91%). ³¹P-NMR (121.4 MHz, CDCl₃): 7.2 ppm. IR (HATR): 2892, 1627, 1437, 1353, 1273, 1158, 1117, 1061, 1032, 1001, 931, 886, 839, 820, 744, 712, 694, 682, 670 cm⁻¹. ES-MS: 844.1 [M-BARF]⁺

Iridium Complex of Ligand Iₚ

In a Schlenk tube under an argon atmosphere, a mixture of ligand (Sₚ)-1-[(1R)-(1-(5,6-methylenedioxy-3H-isobenzofuran-1-ylideneamino)-ethyl)]-2-(diphenylphosphino)-ferrocene (Iₙ). (100 mg, 0.174 mmol) and [Ir(COD)Cl]₂ (58.6 mg, 0.0872 mmol) in dry CH₂Cl₂ (5 mL) was refluxed and stirred during 2 h. After cooling down to room temperature, NaBARF (231.8 mg, 0.262 mmol) was added to the solution and stirred for 5 min. Then, H₂O (5 mL) was added, and the mixture was stirred vigorously for 20 min. The organic layer was seperated, and the aqueous phase is was extracted with CH₂Cl₂ (2×5 mL). The combined organic phases were dried over MgSO₄, filtered and concentrated under reduced pressure. Purification by flash chromatography over silica gel (pentane/CH₂Cl₂, 50/50) resulted in an orange foaming solid, 238.6 mg (80%). ³¹P-NMR (121.4 MHz, CDCl₃): 7.0 ppm. IR (HATR): 2935, 1614, 1506, 1478, 1438, 1353, 1272, 1158, 1117, 1032, 1001, 940, 886, 839, 774, 744, 712, 694, 682, 670, 616 cm⁻¹. ES-MS: 874.1 [M-BARF]⁺

V. Asymmetric Synthesis comprising Imidate Ligands

In another aspect, the present invention provides the use of a catalyst as described above in the synthesis of pharmaceuticals, agrochemicals, flavours and/or fragrances. The catalysts of the invention were found particularly useful in asymmetric synthesis, such as, but not limited to diethylzinc addition to benzaldehyde, aziridination of methyl cinnamate and allylic alkylation. Some examples are provided below.

V.I. Asymmetric Syntheses with Metal Catalysts

Imidate ligands of the invention were examined in the Cu(I)-catalyzed aziridination of alkenes. There are only a few ligand types appropriate for use in the asymmetric Cu(I)-catalyzed aziridination (Ref. 9), the two most important families being bisoxazolines (Ref. 10) and diimines (Ref. 11).

Cu(I)-Catalyzed Asymmetric Aziridination of Methyl Cinnamate

A typical procedure is as follows: Bisimidate (Iₐ) (7.6 mg, 0.022 mmol) and Cu(MeCN)₄PF₆ (7.5 mg, 0.020 mmol) were dissolved in CH₂Cl₂ (2 mL) and stirred for 45 min at room temperature under argon. To this reaction mixture was added 4 Å molecular sieves (100 mg) and methyl cinnamate VIII (162 mg, 1.0 mmol). The resulting suspension was cooled to −40° C. Subsequently, PhINTs (74.6 mg, 0.2 mmol) was added and the reaction mixture was stirred for 21 h. The reaction mixture was passed through a short pad of silica gel and eluted with EtOAc. Evaporation in vacuo and purification by flash chromatography over silica gel (gradient elution with hexane/EtOAc, 90/10 to hexane/EtOAc, 80/20) resulted in IX, 59.3 mg (90%, 45% ee).

The adduct IX was fully characterized by comparison of its spectral data with those reported in the literature. (Ref. 4)

Conditions for chiral HPLC: Chiralcel OD-H column, solvent: n-hexane/EtOH (90/10), flow rate=1 mL/min, T=35° C., retention times: 10.7 min for (2R,3S)-1× and 16.4 min for (2S,3R)-IX.

We observed excellent yields for all bisimidates (Table 4, entry 1 and 3-5) except for imidate I_{b} derived from binaphthyldiamine II_{b} (Table 4, entry 2). With imidate alcohol (I_{f}) and monodentate imidate (I_{g}) as a chiral ligand, low yields were obtained (Table 4, entry 6 and 7). The enantioselectivities were low (Table 4, entry 4-7) to moderate (Table 4, entry 1-3). Nevertheless, the result obtained with ligand Iₐ was promising (Table 4, entry 1): the yield is one of the best found in literature for the Cu(I)-catalyzed aziridination (Ref. 6).

TABLE 4
Cu(I)-catalyzed asymmetric aziridination of methyl cinnamate^{a}

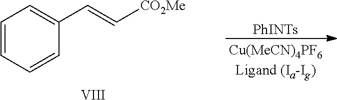

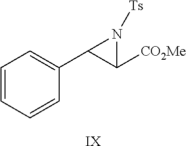

| | | Time | Yield | % | |
|---|---|---|---|---|---|
| Entry | Ligand | (h) | (%)^{b} | ee^{c} | Configuration |
| 1 | Iₐ | 22 | 90 | 45 | 2S, 3R |
| 2 | I_{b} | 21 | 22 | 26 | 2S, 3R |
| 3 | I_{c} | 22 | 90 | 37 | 2S, 3R |
| 4 | I_{d} | 23 | 97 | 3 | n.d. |
| 5 | Iₑ | 21 | 87 | <1 | n.d. |
| 6 | I_{f} | 24 | 24 | 6 | 2S, 3R |
| 7^{d} | I_{g} | 21 | 18 | 11 | 2S, 3R |

^{a}Reagents and conditions: VIII (1 mmol), PhINTs (0.2 mmol), Cu(MeCN)₄PF₆ (10 mol %), ligand I (11 mol %), 100 mg 4 Å mol. sieves, 2.5 mL CH₂Cl₂, T = −40° C.
^{b}Isolated yield, calculated on PhINTs as limiting reagent.
^{c}Determined by HPLC analysis with a chiral stationary phase column (Chiralcel OD—H).
^{d}Because I_{g} is a monodentate ligand, 22 mol % was used.

With ligand Iₐ, optimized reaction conditions were investigated by varying different reaction parameters (Table 5). Changing the copper source resulted in a lower yield and comparable selectivities (Table 5, entry 1-3). With a copper (II) species, the reaction was sluggish and almost no conversion was observed (Table 5, entry 4). Changing the solvent led to very slow reactions (Table 5, entry 5-8). Dichloroethane as a solvent afforded a good yield but lower enantioselectivity than dichloromethane (Table 5, entry 9). The highest enantioselectivity was observed at a temperature of −78° C. (51% ee) (Table 5, entry 11).

TABLE 5
Cu(I)-catalyzed asymmetric aziridination of methyl cinnamate (VII):
Optimization of the reaction parameters with ligand (IIₐ).^{a}

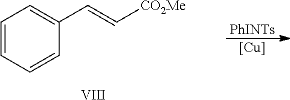

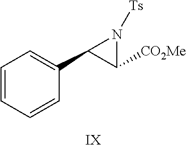

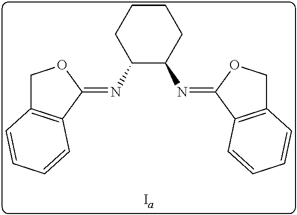

| | | | Temp | Time | Yield | % |
|---|---|---|---|---|---|---|
| Entry | [Cu] | Solvent | (° C.) | (h) | (%)^{b} | ee^{c} |
| 1 | CuOTf | CH₂Cl₂ | −40 | 24 | 18 | 42 |
| 2 | Cu(MeCN)₄BF₄ | CH₂Cl₂ | −40 | 24 | 21 | 42 |
| 3 | Cu(MeCN)₄OTf | CH₂Cl₂ | −40 | 24 | 44 | 46 |
| 4 | Cu(OTf)₂ | CH₂Cl₂ | −40 | 24 | n.d. | — |
| 5 | Cu(MeCN)₄PF₆ | toluene | −40 | 48 | <5 | <1 |
| 6 | Cu(MeCN)₄PF₆ | CH₃CN | −40 | 24 | 31 | 28 |
| 7 | CuOTf | toluene | −40 | 24 | n.d. | — |
| 8 | CuOTf | benzene | 25 | 24 | 56 | 14 |
| 9 | Cu(MeCN)₄PF₆ | (CH₂Cl)₂ | −30 | 24 | 87 | 33 |
| 10 | Cu(MeCN)₄PF₆ | CH₂Cl₂ | 25 | 24 | 64 | 27 |
| 11 | Cu(MeCN)₄PF₆ | CH₂Cl₂ | 78 | 24 | 58 | 51 |

^{a}Reagents and conditions: VIII (1 mmol), PhINTs (0.2 mmol), [Cu] (10 mol %), ligand Iₐ (11 mol %), 100 mg 4 Å mol. sieves, 2.5 mL solvent.
^{b}Isolated yield, calculated on PhINTs as limiting reagent.
^{c}Determined by HPLC analysis with a chiral stationary phase column (Chiralcel OD—H).

### Diethylzinc Addition to Benzaldehyde

The bisimidate ligands were further tested in 1,2-additions of diethylzinc to benzaldehyde. Amino-alcohols are the ligands of choice in this type of reaction. It is known that bisoxazolines without a hydroxyl substituent give low enantioselectivities. (Ref. 7)

A typical procedure is as follows: Bisimidate (II_{b}) (6.0 mg, 0.012 mmol) was dissolved in toluene (2 mL). Et₂Zn (0.75 mL, 1 M in hexane) was added and the resulting yellow solution was stirred for 20 min at room temperature under argon atmosphere. Next, benzaldehyde X (50 μL, 0.49 mmol) was added and the reaction mixture was stirred for another 24 h. The reaction was quenched with 1 mL saturated NH₄Cl solution. The reaction mixture was poured in H₂O (25 mL) and extracted with EtOAc (3×25 mL). The combined organic phases were dried over Na₂SO₄ and evaporated in vacuo. Purification by flash chromatography over silica gel (pentane/EtOAc, 90/10) resulted in XI, 55.7 mg (83%, 75% ee).

Conditions for chiral HPLC: Chiralcel OD-H column, solvent: n-hexane/EtOH (97/3), flow rate=1 mL/min, T=35° C., retention times: 7.8 min for (R)-(+)-XI and 9.0 min for (S)-(−)-XI.

The bisimidate ligands of the invention were compared with bisamidine ligands of the prior art n, o (FIG. 6). (Ref. 8)

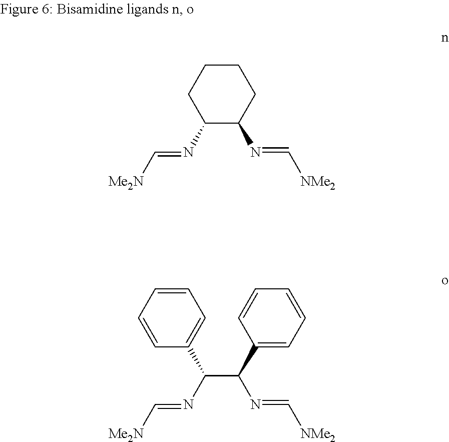

Excellent yields were observed with all bisimidate ligands, except for ligand I_{d} and Iₑ (Table 6, entry 1-5). The monodentate imidate ligands gave slower reactions (Table 6, entry 6-7). Also the bisamidines gave very good yields (Table 6, entry 8-9). However the enantioselectivities were in general low for both bisimidates and bisamidines, with one exception: ligand I_{b} afforded the product in both good yield (83%) and good enantioselectivity (75% ee) (Table 6, entry 2). With ligand I_{b}, we optimized the reaction conditions (Table 6, entries 10-15). Addition of Ti(ⁱOPr)₄ resulted in lower selectivities (Table 6, entry 10-12). Decreasing the temperature resulted in a much slower reaction (Table 6, entry 13). Increasing the amount of ligand resulted in selectivities comparable to our first experiment with ligand I_{b} and a slight decrease in yield (Table 6, entry 14-15).

TABLE 6
Additions of diethylzinc to benzaldehyde in the presence of ligands Iₐ-I_{g}

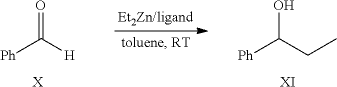

| | | Time | Yield | % | |
|---|---|---|---|---|---|
| Entry | Ligand | (h) | (%)^{b} | ee^{c} | Configuration |
| 1 | Iₐ | 48 | 80 | 11 | S-(−) |
| 2^{d} | I_{b} | 24 | 83 | 75 | R-(+) |
| 3 | I_{c} | 24 | 87 | 14 | R-(+) |
| 4 | I_{d} | 24 | 38 | <1 | n.d. |
| 5 | Iₑ | 24 | 42 | 5 | R-(+) |
| 6 | I_{f} | 24 | 14 | 36 | S-(−) |
| 7 | I_{g} | 24 | 23 | 4 | R-(+) |
| 8 | n | 24 | 87 | 4 | S-(−) |
| 9 | o | 3 | 95 | 24 | S-(−) |
| 10^{d,e} | I_{b} | 24 | 87 | 64 | R-(+) |
| 11^{d,f} | I_{b} | 24 | 73 | 54 | R-(+) |
| 12^{d,g} | I_{b} | 24 | 77 | 46 | R-(+) |
| 13^{d,h} | I_{b} | 72 | 18 | 59 | R-(+) |
| 14^{h} | I_{b} | 48 | 70 | 75 | R-(+) |
| 15^{h,i} | I_{b} | 48 | 71 | 76 | R-(+) |

^{a}Reagents and conditions: X (1 mmol), Et₂Zn (1.5 mmol), ligand (5 mol %), 4 mL CH₂Cl_{2,}the reaction was performed at room temperature.
^{b}Isolated yield.
^{c}Determined by HPLC analysis with a chiral stationary phase column (Chiralcel OD—H).
^{d}2.5 mol % ligand was added.
^{e}2.5 mol % Ti(ⁱOPr)₄ was added.
^{f}20 mol % Ti(ⁱOPr)₄ was added.
^{g}30 mol % Ti(ⁱOPr)₄ was added.
^{h}Reaction temperature = 0° C.
ⁱ10 mol % of ligand I_{b.}

### Palladium(0)-Catalyzed Allylic Alkylation

The bisimidate ligands (Iₐ and I_{b}) together with the mixed phosphino-imidate-ligands (Iⱼ, iₖ, Iₗ and Iₘ) were tested in the palladium(0)-catalyzed asymmetric allylic alkylation. This is a versatile and widely used process in organic synthesis for the enantioselective formation of C—C bonds. First, the allylic substitution of 1,3-diphenyl-2-propenyl acetate (XII) with dimethylmalonate, which is regarded as a standard test substrate for evaluating enantioselective catalysts, was investigated (Table 7).

A typical procedure is as follows: Phosphino-imidate ligand (Iₖ) (12.3 mg, 21.8 μmol) and [Pd(η³-C₃H₅)Cl]₂ (2.0 mg, 5.5 μmol) were dissolved in oxygen-free CH₂Cl₂ (1 mL) and heated for 1 h at 40° C. Next, a solution of rac-1,3-diphenyl-3-acetoxyprop-1-ene XII (55.0 mg, 0.22 mmol) in CH₂Cl₂ (0.5 mL) was added and stirred for another 30 min at room temperature. Finally, a solution of dimethylmalonate (75 μl, 0.66 mmol), BSA (160 μl, 0.66 mmol) and LiOAc (0.7 mg, 10.6 μmol) in CH₂Cl₂ (0.5 mL) was added and the reaction mixture was stirred for 24 h at room temperature. The reaction mixture was passed through a short pad of silica gel and eluted with CH₂Cl₂. Evaporation in vacuo and purification by flash chromatography over silica gel (hexane/EtOAc, 90/10) resulted in XIII, 59.8 mg (85%, 96% ee).

Conditions for chiral HPLC: Chiralcel AD-H column, solvent: n-hexane/EtOH (97/3), flow rate=1 mL/min, T=35° C., retention times: 9.2 min for (S)-XIII and 13.8 min for (R)-XIII.

The results are represented in Table 7.

Bisimidate ligand Iₐ gave no conversion, while ligand I_{b} gave a moderate yield but an excellent enantioselectivity (Table 7, entries 1-2). To our delight, high yields and excellent enantioselectivities were obtained with all imidate-phosphane ligands (Table 7, entries 3-6). The best result was obtained with ligand Iₖ (Table 1, entry 4). We observed also a pronounced N,O-bis-(trimethylsilyl)acetamide (BSA)-activator effect (Table 7, entries 7-9). The enantioselectivity could be further improved when NaOAc was used (Table 7, entry 7). With KOAc and CsOAc as a BSA-activator, almost perfect selectivities and nearly quantitative yields were obtained (Table 7, entries 8-9).

TABLE 7
Asymmetric Allylic Alkylations(AAA)
in the presence of ligands Iₐ-I_{b} and Iⱼ-Iₘ

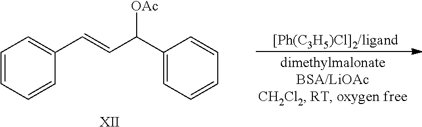

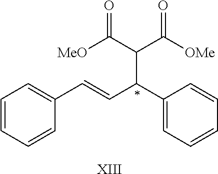

| | | BSA | | | |
|---|---|---|---|---|---|
| | | activator | Yield | % | Config- |
| Entry | Ligand | (h) | (%)^{b} | ee^{c} | uration^{d} |
| 1^{e} | Iₐ | LiOAc | n.c. | — | |
| 2 | I_{b} | LiOAc | 53 | 95 | R |
| 3 | Iⱼ | LiOAc | 82 | 94 | S |
| 4 | Iₖ | LiOAc | 85 | 96 | S |
| 5 | Iₗ | LiOAc | 84 | 96 | S |
| 6 | Iₘ | LiOAc | 85 | 96 | S |
| 7 | Iₖ | NaOAc | 93 | 99 | S |
| 8 | Iₖ | KOAc | 99 | 99 | S |
| 9 | Iₖ | CsOAc | 99 | 99 | S |

^{a}Reaction conditions: XII (0.22 mmol), dimethylmalonate (0.66 mmol), BSA (0.66 mmol), BSA activator (10.6 μmol), [Pd(η³-C₃H₅₎Cl]₂ (5.5 μmol), ligand I (21.8 μmol), CH₂Cl₂ (2 mL), r.t., 16 h.
^{b}Isolated yield.
^{c}Determined by HPLC analysis with a chiral stationary phase column (Chiralcel AD—H).
^{d}Absolute configuration was assigned by the sign of the optical rotation.
^{e}n.c.: no conversion was observed.

To further study the potential of these readily available ligands, other nucleophiles were tested (Table 8). When the reaction was performed with more sterically demanding malonates, excellent yields and selectivities were obtained for the corresponding adducts (Table 8, entries 1-2). Use of dimethyl methylmalonate as a nucleophile and LiOAc as a BSA-activator afforded the corresponding adduct in excellent yield and good enantioselectivity (Table 8, entry 3). By variation of the BSA-activator (Table 8, entries 4-6), the enantioselectivity could be further improved to >99% ee by using NaOAc (Table 8, entry 4). Also acetylacetone was an effective nucleophile in the palladium-catalyzed allylic alkylation reaction: the adduct was formed in 96% yield and with an enantioselectivity of 94% ee (Table 8, entry 7).

TABLE 8
Asymmetric allylic alkylation reactions with various carbon nucleophiles
using Iₖ as an imidate-phosphane ligand.^{[a]}

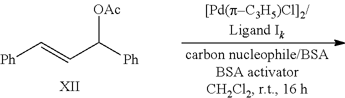

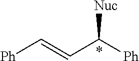

| | Carbon | | | |
|---|---|---|---|---|
| | Nucleophile | BSA- | Yield | ee |
| Entry | (NucH) | activator | [%]^{[b]} | [%]^{[c,d]} |
| 1 | 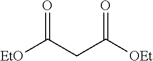 | LiOAc | 98 | 99 (S) |
| 2 | 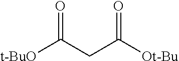 | LiOAc | 81 | 99 (S) |
| 3 4 5 6 | 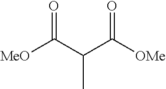 | LiOAc NaOAc KOAc CsOAc | 100 75 100 100 | 79 (R) >99 (R) 94 (R) 82 (R) |
| 7 | 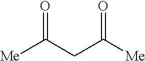 | KOAc | 96 | 94 (S) |

^{a}Reaction conditions: XII (0.22 mmol), carbon nucleophile (0.66 mmol), BSA (0.66 mmol), BSA activator (10.6 μmol), [Pd(η³-C₃H₅)Cl]₂ (5.5 μmol), ligand Iₖ (21.8 μmol), CH₂Cl₂ (2 mL), r.t., 16 h.
^{b}Isolated yield.
^{c}Determined by HPLC analysis with a chiral stationary phase column or with ¹H-NMR using (+)-Eu(hfc)₃.
^{d}Absolute configuration was assigned by the sign of the optical rotation.

Encouraged by the excellent performance of the new imidate-phosphane ligand Iₖ, we studied its potential in the allylic alkylation of the unhindered linear substrate XIV and cyclic substrates XV-XVII. Although highly selective catalysts have been developed for these substrates, they generally exhibit low enantiocontrol in more hindered substrates, such as substrate XII. On the other hand, most catalysts displaying superior enantioselectivities for more hindered substrates like XII behave very poorly for substrates like XIV and cyclic substrates XV-XVII.

FIGURE 7
Comparative example providing an overview of the best results obtained with some of the
most popular ligands in literature for asymmetric allylic alkylations of XII, XIV and XV.

| | 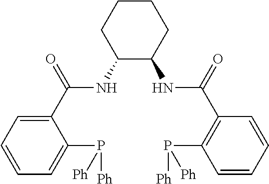 | | 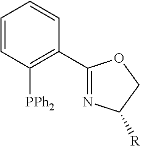 | | 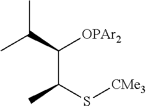 | |
|---|---|---|---|---|---|---|
| | Trost's | | PHOX | | Evans' | |
| | Ligand | | Ligand | | Ligand | |
| XII | 9% | yield | 100% | yield | 28% | yield |
| | 52% | ee | 99% | ee | 96% | ee |
| XIV | 98% | yield | 96% | yield | 94% | yield |
| | 92% | ee | 71% | ee | 91% | ee |

| XV | 86% | yield | — | | — |
|---|---|---|---|---|---|
| | 96% | ee | 0% | ee | |

XII:

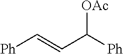

XIV:

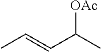

XV:

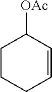

Remarkably, also for the unhindered substrate XIV good enantioselectivities were observed with our catalyst system Iₖ (Table 9, entries 1-5). The best result was obtained when NaOAc was used as a BSA-activator (Table 9, entry 4). For the six-membered cyclic substrate XV, good enantioselectivities were obtained with all BSA-activators (Table 9, entries 6-9). The best results were obtained with KOAc: a good yield was combined with a good enantioselectivity (Table 9, entry 7). We observed a higher selectivity and a quantitative yield for the five-membered cyclic substrate XVI compared to XV (Table 9, entries 10-13). The best result was obtained in the presence of KOAc (Table 9, entry 11). For the seven-membered cyclic substrate XVII an excellent enantioselectivity (90% ee) and yield (100%) was obtained in the presence of NaOAc as a BSA activator (Table 9, entry 16).

TABLE 9
Pd(0)-catalyzed asymmetric allylic alkylation of XIV and XV-XVII with
dimethylmalonate using Iₖ according to an embodiment of the invention
as an imidate-phosphane ligand.^{[a]}

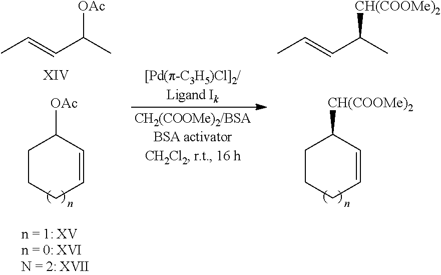

| | | BSA- | Yield | ee |
|---|---|---|---|---|
| Entry | substrate | activator | [%]^{[b]} | [%]^{[c,d]} |
| 1 | XIV | LiOAc | 79 | 78 (S) |
| 2^{e} | XIV | LiOAc | 23 | 80 (S) |
| 3 | XIV | KOAc | 86 | 59 (S) |
| 4 | XIV | NaOAc | 91 | 83 (S) |
| 5 | XIV | CsOAc | 80 | 65 (S) |
| 6 | XV | LiOAc | 49 | 74 (R) |
| 7 | XV | KOAc | 76 | 74 (R) |
| 8 | XV | NaOAc | 31 | 73 (R) |
| 9 | XV | CsOAc | 61 | 73 (R) |
| 10^{[f]} | XVI | LiOAc | 100 | 75 (R) |
| 11^{[f]} | XVI | KOAc | 100 | 86 (R) |
| 12^{[f]} | XVI | NaOAc | 100 | 78 (R) |
| 13^{[f]} | XVI | CsOAc | 100 | 80 (R) |
| 14^{[f]} | XVII | LiOAc | 100 | 87 (R) |
| 15^{[f]} | XVII | KOAc | 100 | 58 (R) |
| 16^{[f]} | XVII | NaOAc | 100 | 90 (R) |
| 17^{[f]} | XVII | CsOAc | 100 | 85 (R) |

^{a}Reaction conditions: XIV-XVII (0.22 mmol), dimethylmalonate (0.66 mmol), BSA (0.66 mmol), BSA-activator (10.6 μmol), [Pd(η³-C₃H₅)Cl]₂ (5.5 μmol), ligand Iₖ (21.8 μmol), CH₂Cl₂ (2 mL), r.t., 16 h.
^{b}Isolated yield.
^{c}Determined by ¹H-NMR analysis by using (+)-Eu(hfc)₃.
^{d}Absolute configuration was assigned by the sign of the optical rotation.
^{e}The reaction was performed at 0° C.
^{f}Complete conversion was obtained within 2 h.

In order to determine whether these excellent results and broad substrate scope were due to the combination of both the chiral ferrocenyl backbone and the imidate nitrogen donor or solely to the presence of the chiral ferrocenyl backbone, we investigated some other nitrogen donors (FIG. 8). When imine-phosphane ligand p was used, we observed a good, but significantly lower enantioselectivity for substrate XII, while with substrates XIV and XV a much lower enantioselectivity was obtained. In addition, when we investigated amidine-phosphane ligand q, which can be considered as the nitrogen analogue of an imidate ligand, both yield and enantioselectivity were much lower as compared to our imidate-phosphane ligand Iₖ. These results show clearly that the presence of the imidate nitrogen donor is required to obtain both high enantioselectivities and a broad substrate scope.

FIGURE 8
Comparison of the imidate-phosphane ligand Iₖ with the imine-phosphane ligand p
and the amidine-phosphane ligand q in the asymmetric allylic alkylation of XII, XIV and XV.

| | 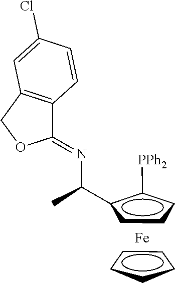 | | 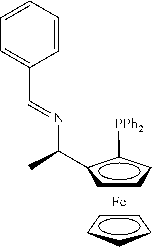 | | 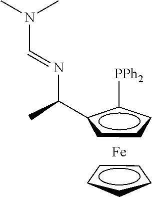 | |
|---|---|---|---|---|---|---|
| | Iₖ | | p | | q | |
| XII | 99% | yield | 94% | yield | 78% | yield |
| | 99% | ee | 91% | ee | 53% | ee |
| XIV | 91% | yield | 69% | yield | 57% | yield |
| | 83% | ee | 69% | ee | 56% | ee |
| XV | 76% | yield | 79% | yield | 37% | yield |
| | 74% | ee | 51% | ee | 27% | ee |

XII:

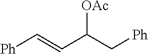

XIV:

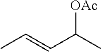

XV:

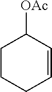

Rarely are enantioselective catalysts successful in both hindered (XII) and unhindered (XIV) or cyclic (XV) substrate classes. Therefore, this imidate-phosphane ligand family can compete with a few other ligands which also provide high selectivities for both hindered and unhindered substrates. Moreover, we have also demonstrated that the presence of the imidate as a nitrogen donor is required to obtain these excellent results.

### Asymmetric Hydrogenations

Enantioselective hydrogenation is one of the most powerful methods in asymmetric catalysis. Although a lot of research has been devoted to this topic, the range of substrates is still limited to certain classes of olefins bearing polar groups which can coordinate with the catalyst. Therefore, the search for new and selective hydrogenation catalysts is still ongoing.

A typical procedure is as follows: Substrate XVIII (0.500 mmol) and iridium(I)-complex of phosphino-imidate ligands Iⱼ or Iₖ (synthesized and isolated prior to reaction, 1 mol %) were dissolved in CH₂Cl₂ (2 mL). The reaction was placed into an autoclave and pressurized to the appropriate pressure with hydrogen. The reaction mixture was stirred at room temperature. After the indicated time, the pressure was released and the solvent was removed in vacuo. The crude product was dissolved in pentane/Et₂O (1:1) and filtered through a short pad of silicagel. Evaporation in vacuo resulted in the hydrogenated product.

Ir(1)-complexes of ligands Iⱼ & Iₖ were tested as catalysts in the hydrogenation of several olefins (Table 10). The best results were obtained with unfunctionalized XVIIId: a perfect conversion and enantioselectivity was observed (Table 10, entries 7-8). Also good to very good results were obtained with other olefin substrates (Table 10, entries 1-6).

TABLE 10
Ir(0)-catalyzed asymmetric hydrogenation of
XVIIIa-d using Iⱼ and Iₖ as an imidate-phosphane ligand.^{[a]}

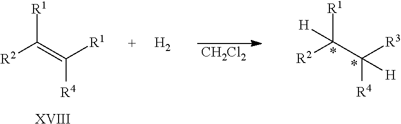

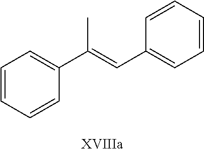

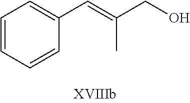

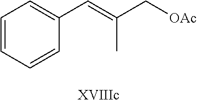

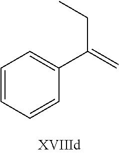

| | | | | Reaction | | |
|---|---|---|---|---|---|---|
| | | | P_{H2} | time | Conversion | Ee |
| Entry | Substrate | Ligand | [bar] | [h] | [%]^{[b]} | [%]^{[c,d]} |
| 1 | XVIIIa | Iⱼ | 50 | 2 | 61 | 71 (−) |
| 2 | XVIIIa | Iₖ | 50 | 2 | 82 | 73 (−) |
| 3 | XVIIIa | Iₙ | 50 | 2 | 98 | 85 (−) |
| 4 | XVIIIa | Iₒ | 50 | 2 | >99 | 91 (−) |
| 5 | XVIIIa | Iₚ | 50 | 2 | 93 | 83 (−) |
| 6 | XVIIIb | Iⱼ | 4 | 2 | 56 | 82 |
| 7 | XVIIIb | Iₖ | 4 | 2 | 40 | 37 |
| 8 | XVIIIc | Iⱼ | 50 | 2 | 88 | 52 |
| 9 | XVIIIc | Iₖ | 50 | 2 | 94 | 70 |
| 10 | XVIIId | Iⱼ | 1 | 2 | 100 | >99 (+)^{[e]} |
| 11 | XVIIId | Iₖ | 1 | 2 | 100 | >99 (+)^{[e]} |

^{a}Reaction conditions: XVIIIa-d (0.500 mmol), iridium-ligand - complex Iⱼ & Iₖ (1 mol %), CH₂Cl₂ (2 mL), r.t., 2 h.
^{b}conversion determined via GC.
^{c}Determined by HPLC analysis with a chiral stationary phase column (Chiralcel OD—H, OJ—H).
^{d}Optical rotations were taken in CHCl₃
^{e}Determined by GC analysis with a chiral stationary phase column (L Chirasil Val).

### REFERENCE LIST

Ref. 1 (a) The Chemistry of Functional Groups: The Chemistry of Amidines and Imidates, Patai S., 1975, 679 pp. (b) Roger, R.; Neilson, D. G. Chem. Rev. 1961, 61, 179-211. (c) Brotherton, T. K.; Lynn, J. W. Chem. Rev. 1959, 59, 841-883.
Ref. 2 Some representative examples are given: (a) Nikokavouras, J.; Papadopoulos, C.; Perry, A.; Vassilopoulos, G. Chimika Chronika 1976, 5, 223-229. (b) Pifferi, G.; Vigevani, A.; Consonni, P.; Gallo, G. G. J. Heterocycl. Chem. 1972, 9, 827-832. (c) Jitsuoka, M.; Tsukahare, D.; Ito, S.; Tanaka, T.; Takenaga, N.; Tokita, S.; Sato, N. Bioorg. Med. Chem. Lett. 2008, 18, 5101-5106. (d) Hall, J. D.; Duncan-Gould, N. W.; Nathan, W.; Siddiqi, N. A.; Kelly, J. N.; Hoeferlin, L. A.; Morrison, S. J.; Wyatt, J. K. Bioorg. Med. Chem. 2005, 13, 1409-1413. (e) Samnes, P. G.; Thetford, D. J. Chem. Soc., Perkin Trans. 1 1989, 3, 655-661. (f) Meyers, A.; Hanagan, M. A.; Trefonas, L. M.; Baker, R. J. Tetrahedron 1983, 39, 1991-1999. (g) Moody, C. J.; Warrellow, G. J. J. Chem. Soc., Perkin Trans. 1 1986, 6, 1123-1128. (h) Dordor, I. M.; Mellor, J. M.; Kennewell, P. D. J. Chem. Soc., Perkin Trans. 11984, 1247-1252.
Ref. 3 Naoki, S. Jpn. Kokai Tokkyo Koho 1988, 12 pp. (JP-87-8553519870407)
Ref. 4 (a) Katsuhiro, K.; Takao, H.; Kazuo, K.; Hiroshi, T.; Makoto, T. Jpn. Kokai Tokkyo Koho 2007, 129 pp. (JP 2006-2882920060206). (b) Birch, A. J.; English, R. J.; Massy-Westropp, R. A.; Slaytor, M.; Smith, H. J. Chem. Soc. 1958, 365-368. (c) Yoshida, H.; Fukushima, H.; Morishita, T.; Ohshita, J.; Kunai, A. Tetrahedron 2007, 63, 4793-4805. (d) Yoshida, H.; Fukushima, H.; Ohshita, J.; Kunai, A. Angew. Chem. Int. Ed. 2004, 43, 3935-3938. (e) Schmidhammer, H. Scientia Pharmaceutica 1981, 49, 34-310.
Ref. 5 Vandyck, K.; Matthys, B.; Van der Eycken, J. Tetrahedron Lett. 2005, 46, 75-78.

Ref. 6 Müller, P.; Fruit, C. Chem. Rev. 2003, 103, 2905-2919.
Ref. 7 Evans, D. A.; Faul, M. M.; Bilodeau, M. T.; Anderson, B. A.; Barnes, D. M. J. Am. Chem. Soc. 1993, 115, 5328-5329.
Ref. 8 (a) Gillespie, K. M.; Sanders, C. J.; O'Shaughnessy, P.; Westmoreland, I.; Thickitt, C. P.; Scott, P. J. Org. Chem. 2002, 67, 3450-3458. (b) Gillespie, K. M.; Crust, E. J.; Deeth, R. J.; Scott, P. Chem. Commun. 2001, 785-786. (c) Sanders, C. J.; Gillespie, K. M.; Bell, D.; Scott, P. J. Am. Chem. Soc. 2000, 122, 7132-7133. (d) Shi, M.; Wang, C.-J.; Chan, A. S. C. Tetrahedron: Asymmetry 2001, 12, 3105-3111. (e) Li, Z.; Quan, R. W.; Jacobsen, E. N. J. Am. Chem. Soc. 1995, 117, 5889-5890. (f) Li, Z.; Conser, K. R.; Jacobsen, E. N. J. Am. Chem. Soc. 1993, 115, 5326-5327.
Ref. 9 Gomez, M.; Muller, G.; Rocamora, M. Coord. Chem. Rev. 1999, 193-195, 769-835.
Ref. 10 (a) Fu, B.; Du, D.-M.; Wang, J. Tetrahedron: Asymmetry 2004, 15, 119-126. (b) Schinnerl, M.; Seitz, M.; Kaiser, A.; Reiser, O. Org. Lett. 2001, 3, 4259-4262.
Ref. 11 Saitoh, A.; Achiwa, K.; Tanaka, K.; Morimoto, T. J. Org. Chem. 2000, 65, 4227-4240.

1. A method of catalyzing a reaction utilizing a cyclic imidate as a ligand for catalysis, the method comprising:
utilizing a catalyst in which the ligand contains substructure (Y) as a minimal structural motive,

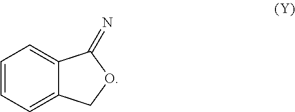

2. The method according to claim 1 wherein the reaction is the synthesis of chiral non-racemic building blocks for pharmaceuticals, agrochemicals, flavors and/or fragrances.

3. The method according to claim 1 wherein the reaction is the synthesis of achiral or racemic building blocks for organic syntheses.

4. The method according to claim 1, wherein the cyclic imidate is a cyclic imidate of
formula (I), or a stereoisomeric form thereof or a salt thereof, 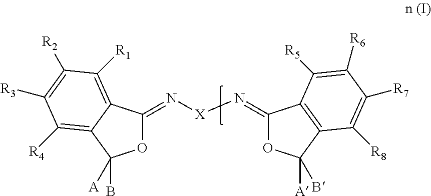
wherein
R1, R2, R3, R4, R5, R6, R7, and R8 are each independently selected from the group consisting of hydrogen, halogen, alkyl, heteroalkyl, aryl, heteroaryl, hydroxyl, amino, diarylphosphanyl, diheteroarylphosphanyl, arylalkylphosphanyl, heteroarylalkylphosphanyl, dialkylphosphanyl; substituted amino, substituted diarylphosphanyl, substituted diheteroarylphosphanyl, substituted arylalkylphosphanyl, substituted heteroarylalkylphosphanyl and substituted dialkylphosphanyl;
A, A′, B, B′ are each independently selected from the group consisting of hydrogen, an alkyl group, a heteroalkyl group, an aryl group, a heteroaryl group, a substituted alkyl group, a substituted heteroalkyl group, a substituted aryl group, and a substituted heteroaryl group;
n is an integer selected from 0 or 1,
wherein when n is 1, X represents a linker connecting both imidate nitrogen atoms via 3 to 8 consecutive bonds; wherein X is selected from the group consisting of alkylene, heteroalkylene, arylene, heteroarylene groups, substituted alkylene, heteroalkylene, arylene, heteroarylene groups, and alkylene, heteroalkylene, arylene, and heteroarylene groups containing one or more heteroatoms;
wherein when n is 0, X represents a linker connecting the imidate nitrogen atom via 3 to 8 consecutive bonds to a chelating substituent excluding a hydroxyl, alkoxy, aryloxy, and amino substituents; wherein X is a substituted group selected from the group consisting of alkyl, heteroalkyl, aryl, and heteroaryl groups.
or wherein when n is 0 and the chelating substituent is R1 excluding a methoxy and chlorine substituents; X represents a group selected from an unsubstituted alkyl, heteroalkyl, aryl and heteroaryl;
or wherein when n is 0, the cyclic imidate of formula (I) is chiral and X represents a heteroatom selected from the group consisting of nitrogen, oxygen, phosphorous and sulfur.

5. A method for the preparation of a compound of formula (I), 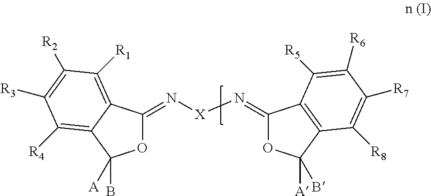
the method comprising:
reacting a compound of formula (II), or a salt thereof, with a reagent of formula X—NH2 (for n=0) or a reagent of formula H2N—X—NH2 (for n=1), wherein 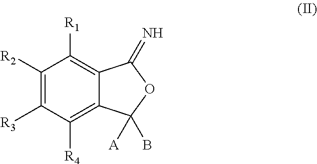
wherein
R1, R2, R3, R4, R5, R6, R7, and R8 are each independently selected from the group consisting of hydrogen, halogen, alkyl, heteroalkyl, aryl, heteroaryl, hydroxyl, amino, diarylphosphanyl, diheteroarylphosphanyl, arylalkylphosphanyl, heteroarylalkylphosphanyl, dialkylphosphanyl; substituted amino, substituted diarylphosphanyl, substituted diheteroarylphosphanyl, substituted arylalkylphosphanyl, substituted heteroarylalkylphosphanyl and substituted diallylphosphanyl;
A, A′, B, B′ are each independently selected from the group consisting of hydrogen, an alkyl group, a heteroalkyl group, an aryl group, a heteroaryl group, a substituted alkyl group, a substituted heteroalkyl group, a substituted aryl group, and a substituted heteroaryl group;
n is an integer selected from 0 or 1,
wherein when n is 1, X represents a linker connecting both imidate nitrogen atoms via 3 to 8 consecutive bonds; wherein X is selected from the group consisting of alkylene, heteroalkylene, arylene, heteroarylene groups, substituted alkylene, heteroalkylene, arylene, heteroarylene groups, and alkylene, heteroalkylene, arylene, and heteroarylene groups containing one or more heteroatoms;
wherein when n is 0, X represents a linker connecting the imidate nitrogen atom via 3 to 8 consecutive bonds to a chelating substituent excluding a hydroxyl, alkoxy, aryloxy, and amino substituents; wherein X is a substituted group selected from the group consisting of alkyl, heteroalkyl, aryl, and heteroaryl groups.
or wherein when n is 0 and the chelating substituent is R1 excluding a methoxy and chlorine substituents; X represents a group selected from an unsubstituted alkyl, heteroalkyl, aryl and heteroaryl;
or wherein when n is 0, the cyclic imidate of formula (I) is chiral and X represents a heteroatom selected from the group consisting of nitrogen, oxygen, phosphorous and sulfur.

6. The method according to claim 5, wherein R1 to R4 equals R5 to R8.

7. The method according to claim 5, wherein n=0 and X is selected from a group consisting of trans-2-hydroxy-1-indanyl, 1-indanyl, [2-(diphenylphosphino)ferrocen-1-yl]-1-ethyl, 2-[(11b)-3H-Binaphtho[2,1-c:1′,2′-e]phosphepin-4(5H)-yl]ethyl and 2-methoxym ethyl-pyrrolidi n-1-yl.

8. The method according to claim 5, wherein n=1 and X is selected from the group consisting of alkyl, trans-1,2-cyclohexadiyl, bis-endo-norbornane-2,5-diyl, or trans-2,2-dimethyl-1,3-dioxolane-4,5-dimethyl or trans-1,2,3,6,7,8-hexahydro-as-indacene-1,8-diyl, aryl and 1,1′-binapht-2,2′-diyl.

9. Cyclic imidate of formula (I) or a stereoisomeric form thereof or a salt thereof, obtained by the process according to claim 5

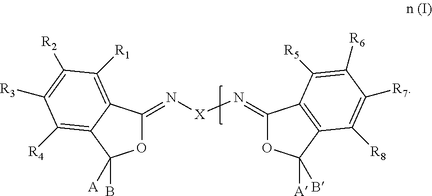

10. Cyclic imidate of formula (I), or a stereoisomeric form thereof or a salt thereof, 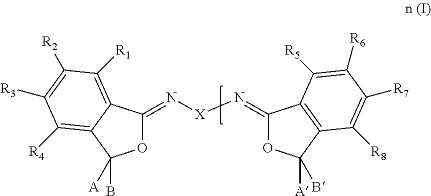
wherein
R1, R2, R3, R4, R5, R6, R7, and R8 are each independently selected from the group consisting of hydrogen, halogen, alkyl, heteroalkyl, aryl, heteroaryl, hydroxyl, amino, diarylphosphanyl, diheteroarylphosphanyl, arylalkylphosphanyl, heteroarylalkylphosphanyl, dialkylphosphanyl; substituted amino, substituted diarylphosphanyl, substituted diheteroarylphosphanyl, substituted arylalkylphosphanyl, substituted heteroarylalkylphosphanyl and substituted dialkylphosphanyl;
A, A′, B, B′ are each independently selected from the group consisting of hydrogen, an alkyl group, a heteroalkyl group, an aryl group, a heteroaryl group, a substituted alkyl group, a substituted heteroalkyl group, a substituted aryl group, and a substituted heteroaryl group,
n is 1, and
X represents a linker connecting both imidate nitrogen atoms via 3 to 8 consecutive bonds; wherein X is selected from the group consisting of alkylene, heteroalkylene, arylene, heteroarylene groups, substituted alkylene, heteroalkylene, arylene, heteroarylene groups, and alkylene, heteroalkylene, arylene, and heteroarylene groups containing one or more heteroatoms.

11. The cyclic imidate of claim 10, wherein R1, R2, R3, R4 have an identical meaning as R5, R6, R7, R8 and A, B have an identical meaning as A′, B′.

12. The cyclic imidate of claim 11, wherein X is selected from the group consisting of alkyl, trans-1,2-cyclohexadiyl, bis-endo-norbornane-2,5-diyl, or trans-2,2-dimethyl-1,3-dioxolane-4,5-dimethyl or trans-1,2,3,6,7,8-hexahydro-as-indacene-1,8-diyl, aryl and 1,1′-binapht-2,2′-diyl.

13. Cyclic imidate of formula (I), or a stereoisomeric form thereof or a salt thereof, 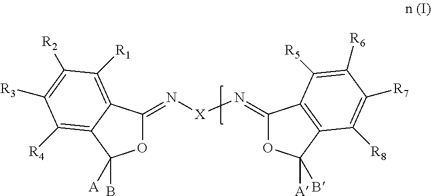
wherein
R1, R2, R3, R4, R5, R6, R7, and R8 are each independently selected from the group consisting of hydrogen, halogen, alkyl, heteroalkyl, aryl, heteroaryl, hydroxyl, amino, diarylphosphanyl, diheteroarylphosphanyl, arylalkylphosphanyl, heteroarylalkylphosphanyl, dialkylphosphanyl; substituted amino, substituted diarylphosphanyl, substituted diheteroarylphosphanyl, substituted arylalkylphosphanyl, substituted heteroarylalkylphosphanyl and substituted dialkylphosphanyl
A, A′, B, B′ are each independently selected from the group consisting of hydrogen, an alkyl group, a heteroalkyl group, an aryl group, a heteroaryl group, a substituted alkyl group, a substituted heteroalkyl group, a substituted aryl group, and a substituted heteroaryl group
n is 0,
wherein when X represents a linker connecting the imidate nitrogen atom via 3 to 8 consecutive bonds to a chelating substituent excluding a hydroxyl, alkoxy, aryloxy, and amino substituents; wherein X is a substituted group selected from the group consisting of alkyl, heteroalkyl, aryl, and heteroaryl groups;
or wherein when the chelating substituent is R1 excluding a methoxy and chlorine substituent; X represents a group selected from an substituted alkyl, heteroalkyl, aryl and heteroaryl;
or the cyclic imidate of formula (I) is chiral and X represents an a heteroatom selected from the group consisting of nitrogen, oxygen, phosphorous or sulfur.

14. The cyclic imidate claim 13, wherein if X represents a linker connecting the imidate nitrogen atom via 3 to 8 consecutive bonds to a chelating substituent, the chelating substituent is not an amide, carboxyl or thiol substituent; or if X represents a heteroatom comprising nitrogen, oxygen, phosphorous or sulfur, the cyclic imidate of formula (I) is chiral non-racemic.

15. The cyclic imidate claim 13, wherein R1, R2, R3 and R4 are hydrogen and
X is selected from a group consisting of trans-2-hydroxy-1-indanyl, 1-indanyl, [2-(diphenylphosphino)ferrocen-1-yl]-1-ethyl, 2-[(11b)-3H-Binaphtho[2,1-c:1′,2′-e]phosphepin-4(5H)-yflethyl and 2-methoxymethyl-pyrrolidin-1-yl.

16. The cyclic imidate of claim 9, wherein the cyclic imidate is a chiral non-racemic compound.

17. A catalyst, wherein the catalyst is formed by complexing a catalyst precursor comprising a 25 metal and a cyclic imidate containing substructure (Y) as a minimal structural motive, wherein the carbon atoms and the nitrogen atom can be optionally substituted by a chemical substituent

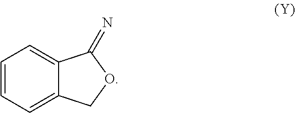

18. The catalyst of claim 17, wherein the cyclic imidate is a cyclic imidate according to any of claims 9 to 15.

19. A method of synthesis of chiral non-racemic building blocks for pharmaceuticals, agrochemicals, flavors and/or fragrances, the method comprising: utilizing the catalyst of claim 17 in the synthesis of chiral non-racemic building blocks for pharmaceuticals, agrochemicals, flavors and/or fragrances.

20. A method of synthesis of achiral or racemic building blocks for organic syntheses, the method comprising: utilizing the catalyst of claim 17 in the synthesis of achiral or racemic building blocks for organic syntheses.

21. The method according to claim 1, wherein any of the carbon atoms and the nitrogen atom is substituted by a chemical substituent.

22. The method according to claim 4, wherein any two of R1, R2, R3, R4, R5, R6, R7, and R5 together with the carbon atom to which they are attached form a carbocyclic fused ring, a heterocyclic fused ring, a substituted carbocyclic ring or a substituted heterocyclic fused ring.

23. The method according to claim 4, wherein A and B, or A′ and B′, together with the carbon atom to which they are attached form a carbocyclic ring, a heterocyclic ring, a substituted carbocyclic ring, or a substituted heterocyclic ring.

24. The method according to claim 5, wherein any two of R1, R2, R3, R4, R5, R6, R7, and R5 together with the carbon atom to which they are attached form a carbocyclic fused ring, a heterocyclic fused ring, a substituted carbocyclic ring or a substituted heterocyclic fused ring.

25. The method according to claim 5, wherein A and B, or A′ and B′, together with the carbon atom to which they are attached form a carbocyclic ring, a heterocyclic ring, a substituted carbocyclic ring, or a substituted heterocyclic ring.

26. The cyclic imidate of claim 10, wherein any two of R1, R2, R3, R4, R5, R6, R7, and R5 together with the carbon atom to which they are attached form a carbocyclic fused ring, a heterocyclic fused ring, a substituted carbocyclic ring or a substituted heterocyclic fused ring

27. The cyclic imidate of claim 10, wherein A and B, or A′ and B′, together with the carbon atom to which they are attached form a carbocyclic ring, a heterocyclic ring, a substituted carbocyclic ring, or a substituted heterocyclic ring.

28. The cyclic imidate of claim 13, wherein any two of R1, R2, R3, R4, R5, R6, R7, and R5 together with the carbon atom to which they are attached form a carbocyclic fused ring, a heterocyclic fused ring, a substituted carbocyclic ring or a substituted heterocyclic fused ring

29. The cyclic imidate of claim 13, wherein A and B, or A′ and B′, together with the carbon atom to which they are attached form a carbocyclic ring, a heterocyclic ring, a substituted carbocyclic ring, or a substituted heterocyclic ring.

30. The cyclic imidate of claim 10, wherein the cyclic imidate is a chiral non-racemic compound.

31. The cyclic imidate of claim 13, wherein the cyclic imidate is a chiral non-racemic compound.
